(19) United States
(12) Patent Application Publication
(10) Pub. No.: US 2024/0226572 A1
(43) Pub. Date: Jul. 11, 2024

(54) PULSE GENERATION AND STIMULATION ENGINE SYSTEMS

(71) Applicant: Presidio Medical, Inc., South San Francisco, CA (US)

(72) Inventors: Michael A. Faltys, San Francisco, CA (US); Aaron Hardinger, Reno, NV (US); James Harris, San Francisco, CA (US); Thomas Flores, San Francisco, CA (US); Douglas Michael Ackermann, Reno, NV (US); Kenneth S. Wu, San Francisco, CA (US)

(21) Appl. No.: 18/563,861

(22) PCT Filed: May 26, 2022
(86) PCT No.: PCT/US2022/031162

§ 371 Date: Nov. 22, 2023

Related U.S. Application Data
(60) Provisional application No. 63/193,557, filed on May 26, 2021.

(51) Int. Cl. A61N 1/36

(52) U.S. Cl. CPC A61N 1/3614; A61N 1/36021; A61N 1/36031; A61N 1/36034; A61N 1/36071; A61N 1/36125

(57) ABSTRACT

A neuromodulation device configured to operate in a plurality of waveform generation modes includes a power source, a control unit in communication with the power source, a bipolar current generator in communication with the control unit and an input of a switching unit, and a plurality of electrodes. Each electrode is in communication with a unique output of the switching unit. The switching unit can provide electrical communication between the bipolar current generator and a selected one of the plurality of electrodes in response to a control signal from the control unit. The current generator can deliver alternating current to the at least one working electrode during a first waveform generation mode and direct current to the working electrode during a second waveform generation mode. An indifferent electrode provides a return path for the alternating current, the direct current, or both. The control unit is configured to detect at least one failure event and prevent, alter, or stop operation of the bipolar current generator in response to the detected at least one failure event.

### BACKGROUND

This application relates, in some embodiments, to facilitating block, modulation or attenuation of biological signals through nerve tissue, including the processing of biological tissue in nervous system tissue, cardiac tissue, or other voltage-sensitive tissue.

The gate control theory of pain was developed in the 1960s and led to the advent of stimulation-based pain management therapies to reduce pain inputs from reaching the brain by selectively stimulating non-nociceptive fibers (non-pain transmitting fibers) in the spinal cord to inhibit transmission of pain stimuli to the brain (See Mendell, Constructing and Deconstructing the Gate Theory of Pain, Pain, 2014 February 155(2): 210-216). Current stimulation systems for spinal cord stimulation (SCS), which act on this gate control theory to indirectly reduce pain, typically have relied on stimulation signals in the <100 Hz frequency range, and recently in the kHz frequency range. Stimulation of the dorsal root ganglia, DRG, in a similar frequency range has also been employed to reduce segmental pain through the same mechanism.

However, technologies based on this premise have drawbacks as pain transmission inhibition is not complete and side effects such as paresthesia can be uncomfortable for patients. Therefore, it is desirable to have systems and methods of treating pain which more effectively block or attenuate pain signal transmission through pain fibers, or decrease the excitability of neurons which process pain signals, rather than indirectly reducing pain signals through gate-theory activation of non-nociceptive fibers, as well as avoid undesirable side effects. Furthermore, block or attenuation of neural tissue or neural activity has been implicated in not only affecting pain but also in the management of movement disorders, psychiatric disorders, cardiovascular health, as well as management of disease states such as diabetes.

### SUMMARY

In one configuration, a neuromodulation device or method with multiple failure modes is provided. In one configuration, a neuromodulation device or method with multiple failure mode detection is provided. In one configuration, a neuromodulation device configured to operate in a plurality of waveform generation modes includes: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; and an indifferent electrode configured to provide a return path for the alternating current, the direct current, or both; wherein the control unit is configured to detect at least one failure event and prevent, alter, or stop operation of the bipolar current generator in response to the detected at least one failure event. In one configuration, a neuromodulation method includes: providing: a power source; a control unit in communication with the power source; a bipolar current generator in communication with the control unit and an input of a switching unit; and a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; and delivering a direct current to the working electrode during a second waveform generation mode; detecting at least one failure event; and preventing, altering, or stopping operation of the bipolar current generator in response to detecting at least one failure event.

The failure event may include an actual stimulating current being unequal to a desired stimulating current, wherein the actual stimulating current is the alternating current or the direct current. The failure event may include a monitored current signal from a power supply to the switching unit exceeding an expected current amount. The failure event may include a real time determination that at least one of a resistance or capacitance of the at least one working electrode is not equal to an expected resistance or expected capacitance of the at least one working electrode. The failure event may include a real time determination that a peak-to-peak voltage of the at least one working electrode is above an expected voltage of the at least one working electrode. The failure event may include a blocking capacitor is not properly functioning.

The direct current may include an anodic current and a cathodic current, and the failure event may include the anodic current not being (1) equal to and (2) opposite in sign to the cathodic current. The direct current may include an anodic current and a cathodic current, and the failure event may include the anodic current not being (1) less than a threshold amount different from and (2) opposite in sign to the cathodic current. The failure event may include any of the failure events described herein.

In another configuration, a neuromodulation device or method with electrode switching unit fault detection is provided. In one configuration, a neuromodulation device configured to operate in a plurality of waveform generation modes includes: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; and an indifferent electrode configured to provide a return path for the alternating current, the direct current, or both; wherein the control unit is configured to monitor a current flowing to a power supply of the switching unit and to deactivate the bipolar current generator when the monitored current violates a threshold condition. In one configuration, a neuromodulation method, includes: providing: a power source; a control unit in communication with the power source; a bipolar current generator in communication with the control unit and an input of a switching unit; and a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; delivering a direct current to the working electrode during a second waveform generation mode; monitoring a current flowing to a power supply of the switching unit; and deactivating the bipolar current generator when the monitored current violates a threshold condition.

Violating a threshold condition may correspond to the monitored current exceeding a threshold level. Violating a threshold condition may correspond to the monitored current falling below a threshold level. The switching unit may include a multiplexer. The neuromodulation device may further include a back-biasing diode in communication with at least one output of the switching unit, wherein the back-biasing diode is configured to prevent back-biasing of the switching unit at least one output.

In another configuration, a neuromodulation device or method with current generator calibration is provided. In one configuration, a neuromodulation device configured to operate in a plurality of waveform generation modes includes: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; an indifferent electrode configured to provide a return path for the alternating current, the direct current, or both; and a bipolar current generator calibration unit comprising a calibration load and a calibration load switch, wherein the control unit is configured activate the calibration load switch to direct current from the bipolar current generation to the calibration load, measure the current directed to the calibration load, and calibrate the bipolar current generator in response to the measured current. In one configuration, a neuromodulation method includes: providing: a power source; a control unit in communication with the power source; a bipolar current generator in communication with the control unit and an input of a switching unit; and a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; delivering a direct current to the working electrode during a second waveform generation mode; providing a bipolar current generator calibration unit comprising a calibration load and a calibration load switch; activating the calibration load switch to direct current from the bipolar current generator to the calibration load; measuring the current directed to the calibration load; and calibrating the bipolar current generator in response to the measured current.

The calibration load may include a resistor. The neuromodulation device may further include a current sensor configured to measure the current directed to the calibration load. Calibrating the bipolar current generator may include adjusting the value of a control signal communicated to bipolar current generator.

In another configuration, a neuromodulation device or method with independent trimming adjustments is provided. In one configuration, a neuromodulation device configured to operate in a plurality of waveform generation modes includes: a power source; a control unit in communication with the power source, wherein the control unit comprises a power controller and a trimming controller; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; and an indifferent electrode configured to provide a return path for the alternating current, the direct current, or both; wherein the power controller is configured to provide a power control signal corresponding to a desired current output level to the bipolar current generator, wherein the trimming controller is configured to provide an adjustment signal to the bipolar current generator, and wherein the current generator is further configured to deliver the alternating current in the first waveform generation mode or the direct current in the second waveform generation mode in response to the power control signal and the adjustment signal. In one configuration, a neuromodulation method includes: providing: a power source; a control unit in communication with the power source, wherein the control unit comprises a power controller and a trimming controller; a bipolar current generator in communication with the control unit and an input of a switching unit; and a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; delivering a direct current to the working electrode during a second waveform generation mode; and determining a power control signal corresponding to a desired current output level to the bipolar current generator; determining an adjustment signal to the bipolar current generator; delivering the alternating current in the first waveform generation mode or the direct current in the second waveform generation mode in response to the power control signal and the adjustment signal.

The bipolar current generator may include at least one amplifier comprising first and second terminals, wherein the first terminal is in electrical communication with the power controller and wherein the second terminal is in electrical communication with the trimming controller.

In another configuration, a neuromodulation device or method with electrocautery and/or defibrillation protection (or other high voltage or current discharge) protection is provided. In one configuration, a neuromodulation device configured to operate in a plurality of waveform generation modes includes: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; an indifferent electrode configured to provide a return path for the alternating current, the direct current, or both; and a protection mechanism configured to prevent electrical damage to the neuromodulation device from energy from an external source. In another configuration, a method includes: providing: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; delivering a direct current to the working electrode during a second waveform generation mode; and preventing electrical damage to one or more of the power source, the control unit, the bipolar current generator, or one or more of the plurality of electrodes from energy from an external source.

The external source may be an electrocautery device or a defibrillator. The protection mechanism may include at least one positive temperature coefficient (PTC) device and a Zener diode. The Zener diode may be electrically in series with at least one PTC device.

In another configuration, a neuromodulation device or method with electrode parameter sensing is provided. In one configuration, a neuromodulation device configured to operate in a plurality of waveform generation modes includes: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; wherein the current generator is configured to deliver alternating current to the at least one working electrode during a first waveform generation mode, and wherein the current generator is further configured to deliver a direct current to the working electrode during a second waveform generation mode; and an indifferent electrode configured to provide a return path for the alternating current, the direct current, or both; wherein the control unit is configured to determine at least one parameter of an electrode in real time and modify operation of the current generator in response to detecting a change in the at least one parameter of the electrode that is greater than a threshold amount. In one configuration, a neuromodulation method includes: providing: a power source; a control unit in communication with the power source; a bipolar current generator in communication with the control unit and an input of a switching unit; a plurality of electrodes, each in communication with a unique output of the switching unit, wherein the switching unit is configured to provide electrical communication between the bipolar current generator and a selected one of the plurality of electrodes in response to a control signal from the control unit; delivering alternating current to the at least one working electrode during a first waveform generation mode; delivering a direct current to the working electrode during a second waveform generation mode; determining at least one parameter of an electrode in real time; and modifying operation of the current generator in response to detecting a change in the at least one parameter of the electrode that is greater than a threshold amount.

The parameter of the electrode may be a series access resistance (Ra) or a double layer capacitance (Cdl). The control unit may be configured to determine the at least one parameter of the electrode in real time by using a test rectangular biphasic current pulse and a potential measured between a working and indifferent electrode. In some embodiments, the techniques and methods described herein may be performed “offline,” or not in real time, using the same techniques, methods and algorithms. The parameter of the electrode may be a series access resistance (Ra) or a cyclic peak-to-peak voltage (Vpp). The control unit may be configured to determine the at least one parameter of the electrode in real time by using a set of points sampled from a potential measured between a working and indifferent electrode and an instantaneous stimulus current. The control unit may be configured to use the at least one parameter of the electrode to control the device according to any of the methods or examples provided herein.

The direct current may include ultra low frequency current. The ultra low frequency currents may be less than about 5 Hz, less than about 2 Hz, or less than about 1 Hz. The alternating current may be high frequency alternating current. The the high frequency alternating current may be at least about 1 kHz, or between about 5 Hz and about 1 kHz.

The power source may include a battery. The control unit may include a first control unit and a second control unit configured to run independent algorithms. The neuromodulation device may be configured to measure an offset current when the neuromodulation device is in the second waveform generation mode. The neuromodulation device may be configured to measure cyclic Vpp of the at least one working electrode. The neuromodulation device may also include a virtual ground configured to be operably connected to the indifferent electrode where the virtual ground can be set to any level to minimize power dissipation.

### BRIEF DESCRIPTION OF THE DRAWINGS

FIG. 1 illustrates a schematic of application-specific integrated circuit (ASIC).

FIG. 2 illustrates a schematic of an ASIC with a DC and AC discrete approach.

FIG. 3 illustrates a schematic of an ASIC adapted to deliver DC.

FIG. 4 illustrates a schematic of an ASIC adapted to deliver DC with a current source.

FIGS. 5 and 5B illustrates schematics of systems with a current source.

FIG. 6 illustrates a schematic of a fail-safe hybrid system.

FIG. 7 illustrates a table of example mechanism and mitigations.

FIG. 8 illustrates a graph relating to lost capacity and voltage protections.

FIG. 9A illustrates a graph relating to the disconnection of a reference electrode.

FIG. 9B illustrates a graph relating to bias removal.

FIG. 10 illustrates a graph of stimulation startup against time in seconds.

FIG. 11 illustrates a schematic relating to HW and FW fail-safe.

FIG. 12 illustrates a schematic of an example stimulation engine that can provide ULF and AC simulation with a single architecture.

FIG. 13 illustrates one embodiment of a system for protecting against high voltage and/or high current using steering diodes and fast-acting positive temperature coefficient devices.

FIG. 14 illustrates the system of FIG. 13 clamping a dangerous high voltage to a safe voltage less than 20 V.

FIG. 15 illustrates a graph of a measurement of an ultra low frequency (ULF) waveform (sometimes referred to herein as a DC waveform) stimulating a ULF lead in-vitro, which may be generated by the stimulation engine of FIG. 12 operating in DC mode.

FIG. 16 illustrates a graph of an example AC waveform, which may be generated by the stimulation engine of FIG. 12 operating in AC mode.

FIG. 17 illustrates a table of mitigation mechanisms that may be included in the stimulation engine of FIG. 12.

FIG. 18 illustrates a square wave inserted at the zero crossing point of an ultra low frequency (ULF) waveform to enable real-time measurement and calculation of circuit parameters.

FIG. 19 illustrates a method for real-time measurement and calculation of circuit parameters by utilizing a range of approximate values for circuit parameters and a peak and/or slope calculation.

FIG. 20 illustrates a method for real-time measurement and calculation of circuit parameters by sampling current and voltage at limited points of the ultra low frequency waveform.

FIG. 21 illustrates a method for real-time measurement and calculation of circuit parameters by sampling current and voltage at limited time points of an alternating current waveform.

FIG. 22 illustrates a schematic of a reference electrode selector.

FIG. 23 illustrates a schematic of various instrumentation amplifiers that may be used to measure various voltages of the stimulation engine of FIG. 12.

FIG. 24 illustrates a schematic of various features, such as a current generator(s), rebalance switch, current setting, and polarity/zero, among others of the stimulation engine of FIG. 12.

FIG. 25 illustrates the arrangement of FIGS. 25A and 25B.

FIGS. 25A and 25B illustrate a schematic of an adjustable stim supply.

### DETAILED DESCRIPTION

This application relates, in some embodiments, to internal and external pulse generation and/or stimulation engine systems for facilitating block, modulation and/or attenuation of biological signals through nerve tissue, including the processing of biological tissue in nervous system tissue (including but not limited to neurons and glial cells), cardiac tissue, or other voltage-sensitive tissue. In some embodiments, either the anodic or cathodic phases of a delivered waveform to a patient, or both the anodic and cathodic phases can have a therapeutic effect on electrically excitable tissue, such as neural tissue for example.

In some embodiments, a pulse generation and/or stimulation engine system comprises any one or more of the features described in the disclosure.

In some embodiments, a pulse generation and/or stimulation engine method comprises any one or more of the features described in the disclosure.

Conventional stimulation systems can utilize capacitors to guarantee or promote fail-safe operation because they are reliable and low cost.

Some systems cannot use capacitors because they are fully integrated on silicon, the output frequencies are too low and capacitors would be too large, or some systems must pass direct current (DC). Some embodiments of systems can operate by providing a low frequency AC (LF-AC) waveform in conjunction with a low-level DC bias for the purpose of keeping the electrode operating range within a voltage window. The safety mechanisms in essence assure that both components stay within specification and that the resulting electrode voltages stay within the prescribed range as evaluated by, for example, at least two independent checking mechanisms. As for traditional and high frequency AC, capacitors can be switched in-line to protect against DC, and protection against switch failure can be afforded by assuring virtually no DC passes through the can, the only single-fault path that DC can take.

Disclosed herein, in some embodiments, are alternative embodiments to capacitors to increase patient safety and/or combined use of capacitors to provide protection, and in some cases for higher frequencies only.

Not to be limited by theory, the propagation of action potentials in electrically excitable tissue, e.g., neural tissue, leads to refractory periods on the order of milliseconds for sodium channels, typically between about 1 ms and about 20 ms, or between about 2 ms and about 5 ms for the combined absolute and relative refractory periods, thus very low frequency AC current waveforms with half periods meaningfully greater than this refractory period (e.g., greater than about 1 ms, 1.5 ms, 2 ms, 2.5 ms, 3 ms, 10 ms, 30 ms, 50 ms, 100 ms, 300 ms, 500 ms, 1000 ms, 2000 ms, 5000 ms, 6000 ms or more) and have sufficiently low differential rates (e.g., rise and fall-times) to not induce action potentials can also be used to create tissue blockade or attenuation, and will be perceived by electrically excitable tissue as a direct current stimulus. As such, direct current (DC) as defined herein is inclusive of low frequency AC current waveforms that are perceived as and functionally is direct current from the perspective of the tissue whose action potentials or neural processing are being modulated. Indeed, the terms DC, DC waveform, low frequency AC, ultra low frequency AC, ULF, ULF waveform, etc. as used herein may all refer to the same signal, such as any signal or waveform that is perceived by tissue as a DC signal during at least part of the signal or waveform period. The frequency of such waveform could be, for example, less than about 20 Hz, 10 Hz, 9 Hz, 8 Hz, 7 Hz, 6 Hz, 5 Hz, 4 Hz, 3 Hz, 2 Hz, 1 Hz, 0.5 Hz, 0.1 Hz, 0.05 Hz, 0.01 Hz, 0.005 Hz, 0.0001 Hz, or ranges including any two of the foregoing values so long as the direction of current flow is constant over at least the entire refractory period of the target tissue, or at least twice as long, or at least five times as long, or at least ten times as long as the refractory-causing membrane channel time constant (for example, fast sodium channel inactivation gate time constant).

In some embodiments, systems and methods can incorporate a variety of waveform frequencies, including high frequencies, e.g., about 1.2-50 kHz or higher; conventional frequencies, e.g., between about 20-1.2 kHz; low frequencies, e.g., between about 2-20 Hz; and ultra-low frequencies, e.g., below about 2 Hz. As noted elsewhere herein, direct current as defined herein is inclusive of low frequency AC current waveforms that are perceived as and functionally is direct current from the perspective of the tissue whose action potentials are being modulated.

Chronic pain is a significant burden on individuals and society as a whole. Nearly 50 million adults are estimated to have significant chronic or severe pain in the US alone. (See Nahin, Estimates of Pain Prevalence and Severity in Adults: United States, 2012, The Journal of Pain, 2015 Aug. 16(8): 769-780). Worldwide, chronic pain is estimated to affect more than 1.5 billion people. (Borsook, A Future Without Chronic Pain: Neuroscience and Clinical Research, Cerebrum, 2012 June). While surgical techniques are sometimes applied to remove a specific source of pain, frequently due to impingement of a nerve, in many cases the precise cause of pain is not clear and cannot be reliably addressed via a surgical procedure. Pain management can alternatively be addressed by overwhelming the central nervous system with stimulating signals that prevent registration of pain inputs (gate control theory of pain). Typically, this stimulation in the case of spinal cord stimulation (SCS) is performed using metal electrodes and alternating current (AC) stimulation to produce these additional stimulating signals to prevent pain sensation. However, one major drawback is the presence of paresthesia, a sensation of tingling in the innervated region downstream from the stimulated nerve. Methods to eliminate paresthesia which patients can find discomforting have led to different means of stimulation from conventional tonic SCS (˜30-120 Hz) stimulation including high frequency stimulation (˜10 kHz) and burst stimulation (e.g., five pulses at 500 Hz delivered 40 times per second). (Tjepkema-Cloostermans et al. Effect of Burst Evaluated in Patients Familiar With Spinal Cord Stimulation, Neuromodulation, 2016 Jul. 19(5):492-497).

An alternative means to manage pain signaling to the central nervous system is to prevent conduction of the pain signals from the peripheral signal source by directly blocking or attenuating the pain signals as compared to masking the pain signals by generating alternative neural inputs to crowd out and inhibit pain signal transmission as in traditional SCS and gate theory. One means to do this is by applying a direct current (DC) to a nerve to prevent action potential (AP) generation and transmission. Because this does not stimulate the nerve as in traditional stimulation, paresthesia can be avoided. The mechanism leading to AP block has been attributed to a depolarization block or hyperpolarization block that deactivates the sodium channels required for an action potential event under the electrode site. (See Bhadra and Kilgore, Direct Current Electrical Conduction Block of Peripheral Nerve, IEEE Transactions on Neural Systems and Rehabilitation Engineering, 2004 Sep. 12(3): 313-324). Wide dynamic range (WDR) neurons integrate pain signals and have also been implicated as a contributing source of pain in patients, and application of direct current (DC) is well positioned to reduce this activity and may impact associated inhibitory and excitatory neurons that drive WDR activity.

The unmitigated use of direct current has long been known to be dangerous to nerve tissue due to creation of toxic species at the electrode-nerve interface. As such, systems and methods that facilitate safe delivery of direct current therapy would be highly desirable. In some embodiments, systems and methods can be configured to treat nociceptive pain. In some embodiments, systems and methods of treating pain and other medical can involve selective blockade of antero-lateral column tissue in the spinal cord. Furthermore, some embodiments relate to systems and methods of treating pain by the aforementioned systems and methods, specifically through selective blockade of dorsal root tissue and/or dorsal root ganglia. Moreover, in some embodiments, disclosed herein are systems and methods of treating pain, specifically through blockade or attenuation of one or more peripheral nerves.

In some embodiments, systems and methods can safely block or attenuate pain signals (which includes modulation of pain processing) in the spinal column by delivering very low frequency stimulation in the epidural space for up to two weeks or more, to achieve clinically measurable pain reduction in patients with chronic low back pain who are candidates for spinal cord stimulation (SCS).

With targeted nerve block, pain from specific dermatomes and pain in regional body sites can be managed. A number of localized targets implicated in moderating pain signal transduction can be addressed. For example, both more centrally located nerve tissues such as the spinothalamic tract and dorsal root ganglion can be targeted to manage lower back pain, sciatica, and complex regional pain syndrome (CPRS) among other pain considerations.

In some embodiments, an electrode can include a contact comprising a high charge-capacity material. The electrode contact can have in some cases a geometric surface area of between about 1 mm² and about 10 mm², or about 1 mm², 2 mm², 3 mm², 4 mm², 5 mm², 6 mm², 7 mm², 8 mm², 9 mm², 10 mm², 20 mm², 50 mm², 100 mm², or ranges including any two of the foregoing values. The electrode contact itself can be fabricated of a high charge capacity material, such as those described, for example, in U.S. Pat. No. 10,071,241 to Bhadra et al., which is hereby incorporated by reference in its entirety. Alternatively, the electrode contact can comprise a base at least partially, or entirely coated with a high charge capacity material. In some embodiments, a high charge capacity material can have a Q value of at least about 25, 50, 100, 200, 300, 400, 500, 1,000, 2,500, 5,000, 10,000, 50,000, 100,000, 500,000, or more μC, or ranges including any two of the foregoing values. The Q value of an electrode contact can refer to the total amount of charge that can be delivered through an electrode contact before the electrode contact begins generating irreversible chemical reactions at a rate that cannot be cleared through the body's nominal transport mechanism. These chemical reactions include but are not limited to oxygen or hydrogen evolution, or dissolution of the electrode materials. Non-limiting examples of high charge capacity materials are platinum black, iridium oxide, titanium nitride, tantalum, silver chloride, poly(ethylenedioxythiophene) and suitable combinations thereof. The electrodes can comprise fractal coatings or high surface area formats in some embodiments. High charge capacity materials may be configured to be monolithic or as coatings on base substrates. Non-limiting examples of substrates for coating include stainless steel such as 304 and 316LVM, nickel-cobalt-chrome alloys such as MP35N®, platinum and platinum-iridium, titanium, nickel-titanium alloys such as Nitinol. In some embodiments, the electrodes can include tantalum coated with titanium nitride. Tantalum as one non-limiting example can be a particularly advantageous material for its superior radiopacity, thus allowing for improved implantation, verification, and/or removal of implantable neuromodulation devices. In some embodiments, the electrodes can include one or more of titanium nitride, tantalum, and MP35N. To generate more surface area for the electrochemical reactions to occur, the traditional electrodes may be made from high surface area to volume structures such as roughened surfaces, woven surfaces, patterned surfaces, reticulated foam structures, porous sintered bead structures, nano- or micro-patterned structures to expose additional material surface area. In some embodiments, the electrode can be a SINE (separated-interface nerve electrode) or EICCC (electron to ion current conversion cell) electrode in which an electrode is immersed in an electrolyte solution which is in contact with an ion-conductive material-electrolyte solution interface with an ion-conductive material that electrically contacts the cardiac tissue or area proximal to cardiac tissue, as described, for example, in U.S. Pat. No. 9,008,800 to Ackermann et al., and U.S. Pub. No. 2018/0280691 to Ackermann et al., which is hereby incorporated by reference in their entireties.

In some embodiments, disclosed herein are systems and methods for safely and efficaciously stimulating neural tissue that can advantageously utilize a variety of waveforms from DC to high frequencies. Stimulation with DC, although potentially very useful, has not been commercially utilized for neural modulation because neurostimulation systems capable of delivering DC safely for long periods of time have not been available. Available commercial systems prevent DC delivery to limit irreversible electrochemical reactions, relying on charge balancing mechanisms. These systems can include blocking the DC component with capacitors, blocking capacitors, or mechanisms that remove charge accumulation at the end of a stimulation cycle. While reliable, typical capacitors limit charge to less than about one millicoulomb (mC) per phase, disallowing the use of ultra-low frequency signals at large charge magnitudes in excess of this charge capacity. The other widely utilized technique relies on actively balanced current sources, but these require redundancy to be fault tolerant and typically do not deliberately control electrode voltages important for some electrode technologies and have not been shown to be advantageous for long-term high charge delivery. Active systems in conjunction with coatings have been utilized in such devices as retinal implants to increase charge densities to about ˜2 mC/cm², but these densities are still insufficient to allow use of very high charge per phase waveforms required by DC or very low frequency waveforms with sufficient current amplitude.

Some embodiments involve high surface area electrode coatings in conjunction with a bias current such as, for example, a DC bias to maintain the electrode voltages in the optimal range for a particular electrode material for long term operational durability. This approach can boost the charge per phase from about 50 μC/cm² used in conventional systems to about or at least about 5,000 μC/cm², 25,000 C/cm², 50,000 μC/cm², and beyond in some cases without, for example, causing damage to either the electrode or the electrically excitable tissue. Systems and methods configured to allow for an intentional net bias current, e.g., DC bias, such as via a control system, can, in some cases, advantageously maintain the health of the high charge capacity electrodes (by preventing or inhibiting corrosion, e.g., oxidation, or other damage to the electrodes) as well as minimizing or preventing undesired reactions and generation of species such as OH—, H+ or oxygen free radicals that can lead to tissue damage. In some embodiments, the charge per anodic and/or cathodic phase is, for example, about 3,000 μC, 3,500 μC, 4,000 μC, 4,500 μC, 5,000 μC, 5,500 C, 6,000 μC or more or less, such as between about 4,000 μC and about 5,000 μC per phase, and ranges including any two of the foregoing values.

In some embodiments, systems and methods for the delivery of current via implanted electrodes do not include capacitors such as blocking capacitors. In some embodiments, systems and methods for the delivery of current via implanted electrodes do not include resistors.

In some embodiments, the bias current is the current resulting from the summation of the currents being simultaneously delivered to the electrode contacts or working electrodes in proximity to the target excitable or voltage-sensitive tissue. In some embodiments, the bias current is equal in magnitude and opposite in polarity to the summation of the currents being simultaneously delivered to the electrode contacts or working electrodes. In some embodiments, the currents being simultaneously delivered to the electrode contacts or working electrodes can be adjusted to modulate the bias current.

In some embodiments, conventional AC systems, which can include an AC exclusive system, utilizes a capacitor on each/every output, e.g., electrode, to prevent delivery of DC to tissue. Conventional AC systems typically do not include bypass switches that can circumvent the capacitors, which may be needed for direct current (including at ultra-low frequencies as noted above, for example) waveform delivery.

In some embodiments, disclosed herein is a neuromodulation device configured to perform in multiple electrical modulation modes with a single architecture. The device can include, for example, a power source; a control unit; and/or one or more current generators (e.g., monopolar and/or bipolar) configured to be connected to at least one, two, three, four, or more working electrodes.

In some embodiments, a device can include stimulation circuitry including at least one, two, or more blocking capacitors configured to block direct current, at least one, two, or more indifferent electrode switches configured to be in electrical communication with at least one, two, or more indifferent electrodes, and at least one, two, or more blocking capacitor switches in electrical communication to bypass the at least one, two, or more blocking capacitors.

The device can include a first stimulation mode in which the current generator is configured to deliver alternating current to the at least one working electrode, and a second stimulation mode in which the current generator is configured to deliver direct current to the at least one working electrode, both return electrodes absorbed through the indifferent electrode,

In some embodiments, in the first stimulation mode the control unit configures another current generator to route though a second working electrode and causes the at least one indifferent electrode switch to disable the electrical communication between the current generator and the at least one indifferent electrode, and at least one blocking capacitor is active to block direct current.

In some embodiments, in the second stimulation mode the two current generators are configured such that an offset current from, for example, 0 μA to a 1,000 μA or more is configured to pass through the indifferent electrode switch toward the indifferent electrode, and the control unit causes the at least two blocking capacitor switches to disable the electrical communication between the current generator at the at least one blocking capacitor, thereby bypassing the at least two blocking capacitors.

In some embodiments, a device can be configured such that alternating current of ultra low, conventional, or high frequencies can be delivered from a current generator to any number of working electrodes, while an anodic or cathodic bias current is delivered to any number of working electrodes, with the blocking capacitor switches configured to bypass the blocking capacitors, which can be advantageous for, for example, electrode longevity.

In some embodiments, application-specific integrated circuits (ASICs) including some embodiments herein are configured for low power, highly versatile AC stimulation. Some embodiments can add DC but may not necessarily be optimal for DC because the DAC (digital to analog converter) resolution is relatively low, limiting DC bias/offset selectivity (DC offsets can be, for example, as low as 1 μA while simultaneously providing stimulation currents as high as, for example, 25 mA on the same channel), and the power while running in DC mode is relatively high because the current may be on continuously (100% duty cycle) or substantially continuously whereas conventional AC stimulation pulses may be on 250 μs every 25 ms (1% duty cycle).

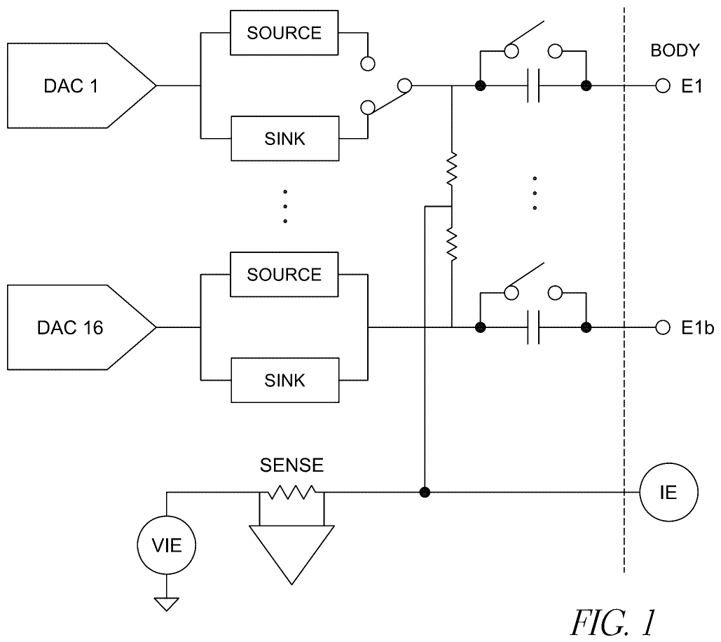

In some embodiments, in reference to FIG. 1, a DC and AC approach, using an application-specific integrated circuit (ASIC), can include a bypass switch around each output blocking capacitor such that current, e.g., DC can be delivered when the bypass switch is closed and charge balanced AC can be delivered when the bypass switch is open. This can be applied to each channel, such as, for example, channels 1 and 16. Accordingly, if the bypass switch fails and current flows due to imbalance in current sources to the tissue due to a short in the switch (or other cause), a “sense” safety mechanism (“sense” circuit shown in FIG. 1) can be used to detect excess current flowing through the system back through the indifferent electrode, e.g., IPG can, and then shut off the stimulation and/or take another safety action measure.

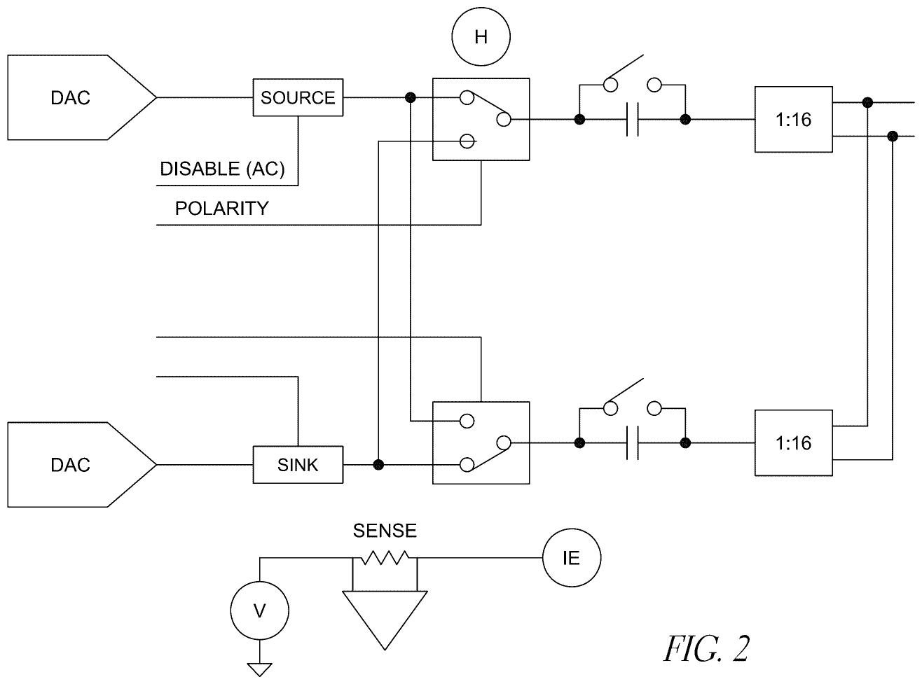

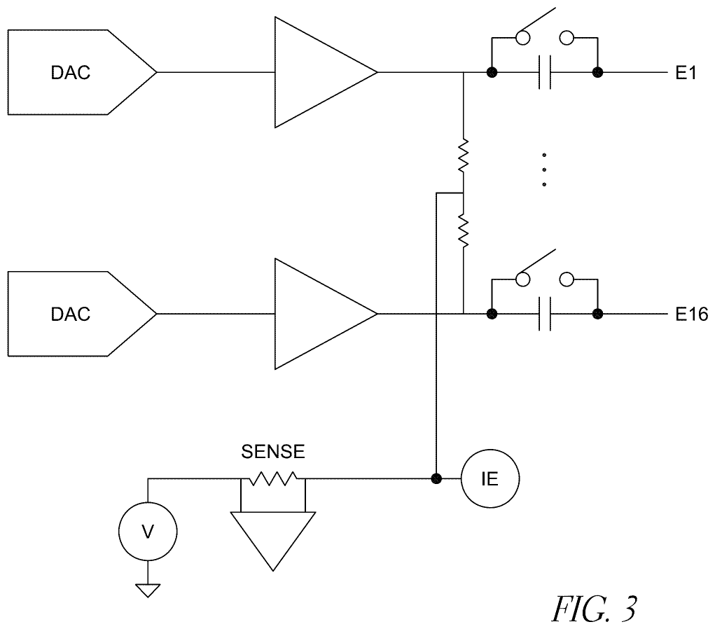

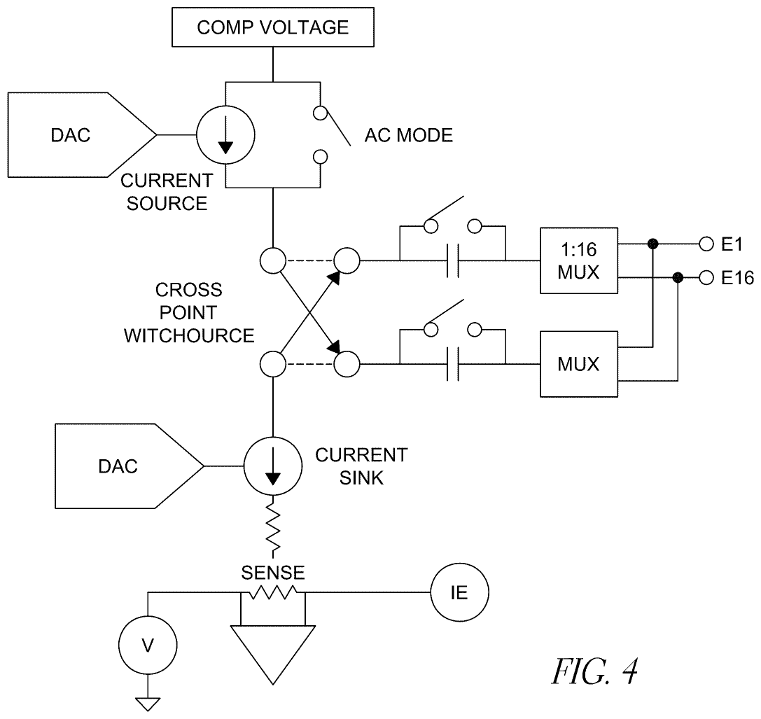

In some embodiments, in reference to FIG. 2, a DC and AC discrete approach can be implemented with fewer components. For example, a blocking capacitor and bypass switches can be reduced to a single set between the source and the multiplexer (mux), which directs the current to a number of channels, such as for example 1 of 16 channels. In some embodiments, more than one set is used. In some embodiments, more or less than 16 channels can be used and/or the current can be directed to one or more channels. A second source and mux can also be used to sink or source the current for bipolar operation. This discrete approach can be advantageously simplified, which can reduce size, cost, manufacturing complexity, etc., by not including a capacitor and switch for each mux output (electrode). When the discrete approach described above and shown in FIGS. 3-4 is not implemented, a capacitor and switch for each channel may be needed (for example, 16 capacitor and switch pairs for a 16 channel system may be needed). A “sense” safety mechanism and IE (an indifferent electrode, or device housing or “can”) can be used, as described in reference to FIG. 2, in the DC and AC discrete approach as well.

FIG. 3 schematically illustrates an embodiment utilizing linear current generators (e.g., Howland Current Pumps) of an ASIC adapted to deliver both DC and AC. DACs can directly control the current generators (sources and sinks) and external capacitor bypass switches have been included in the system to support DC. Because bypass switches, e.g., silicon bypass switches are exposed to the body, safety can be ensured, shutting down if any DC current is detected through the indifferent electrode (IE)/can.

Alternatively, both AC and DC systems can be implemented with a discrete system sharing common components. Some embodiments include a single bipolar channel that can be configured across any pair of electrodes in the system, such as 16 electrodes in some cases. The AC system can be configured to only include a single current sink and can be configured to be sufficiently fast to produce, in some embodiments, about 10 μs pulses requiring slew rates of about 10 V/μs. This current source can be first routed through a cross point switch to alternate polarities across a set of capacitors that are then routed through a multichannel multiplexer, such as a 1 to 16 channel multiplexer for example. A single set of capacitors rather than a capacitor on each electrode can be used because safety again is confirmable by detecting DC current through the IE/CAN.

The AC Discrete Architecture embodiments as described above for example can be extended to handle DC with the addition of a current source, such as schematically illustrated in FIG. 4. To save power, this current source can be extremely slow requiring for example, about, or less than about 5, 4, 3, 2, or 1 V/ms rather than a higher amount, such as, for example, 10 V/μs or more required by the current sink. In addition, switches can be added to bypass the capacitors that may be absent with DC configurations. DC generally requires higher current resolution than AC to be able to accurately set the bias/offset current. As such, in some embodiments, the DAC resolution on both the current source and sink should be less than about 10, 9, 8, 7, 6, 5, 4, 3, 2, or 1 μA, or less than about 5 μA in some cases.

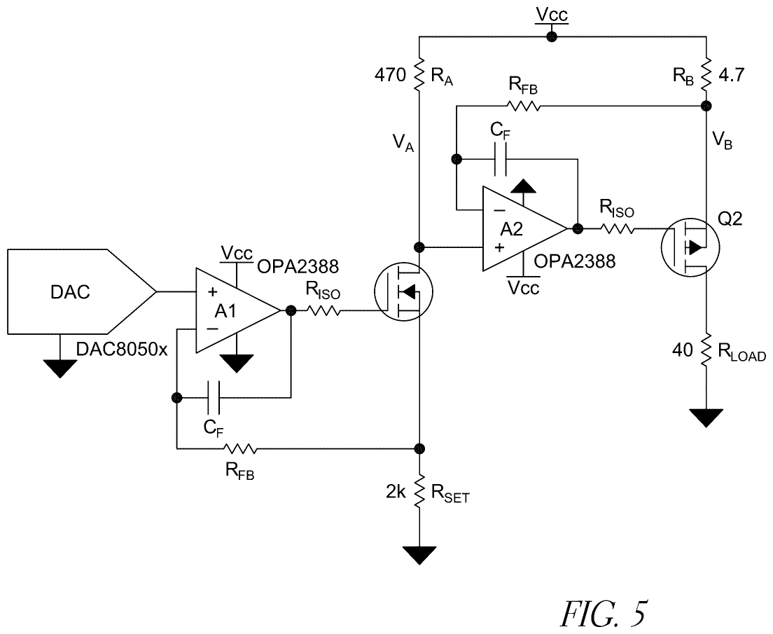

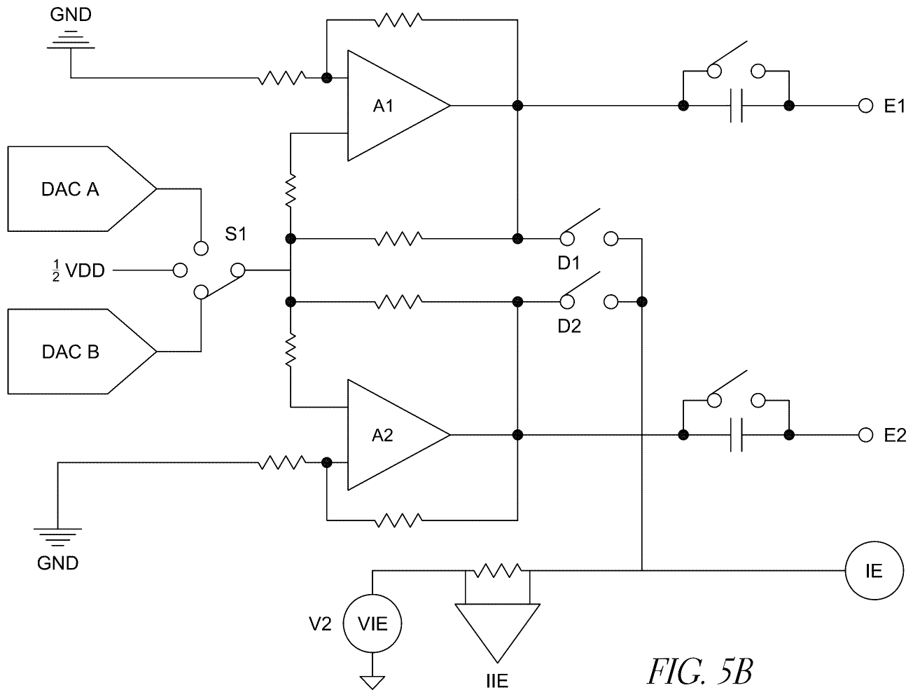

With respect to current sinks and current sources, in some cases, discrete current sinks can be simple and inexpensive to implement. A current source may in some cases be more involved and a circuit similar to that shown in FIGS. 5A-5B) may be needed. Whereas the current sink implements AC and generally needs to be high performance, the current source generally only handles low speed DC and can operate much more slowly, advantageously reducing power significantly.

As described herein, some stimulation systems utilize capacitors to guarantee near fail-safe operation because they are passive, low cost, and generally reliable components. Some systems, however, cannot use (or it is at least less desirable to use) capacitors because they are fully integrated on silicon, the output frequencies can be too low, the system can be capable of passing DC, and/or capacitors can be too large.

FIG. 5B is a schematic of a bipolar current generator that can be dynamically reconfigured to generate AC or DC stimulation (also referred to as operating in AC mode or DC mode). DAC A and B provide the stimulus amplitude. For slow moving DC stimulation DAC A and B are updated slowly (e.g., 100 Hz or less) with the bias added into each current. For AC stimulation, DAC A and B are updated with the value of the activation and recovery current and S1 is switched to form fast AC activation and recovery pulses. Discharge switches are available to create other AC modes such as passive recharge.

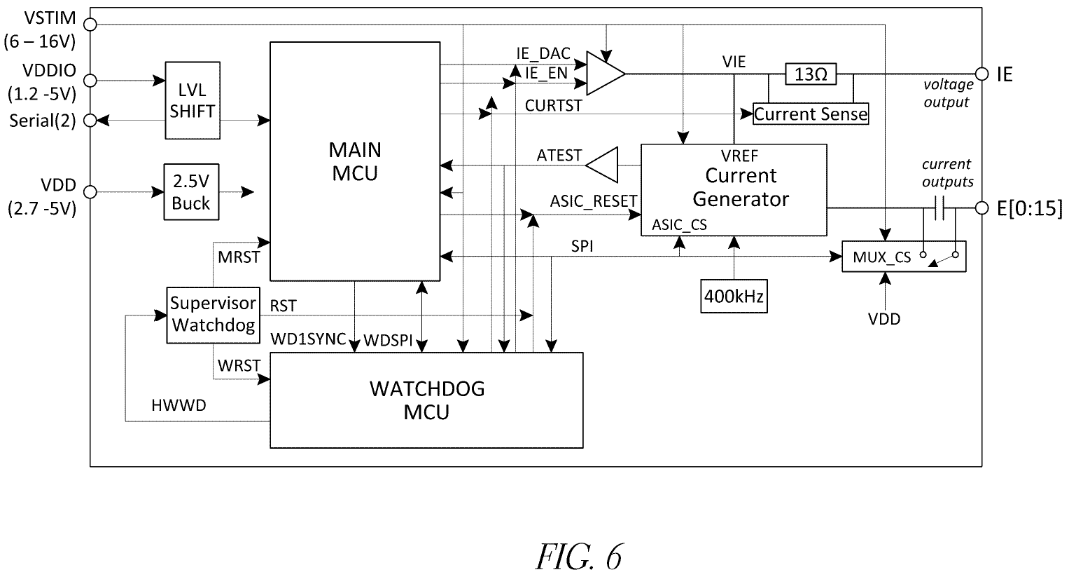

FIG. 6 schematically illustrates a block diagram of a fail-safe hybrid system. The system can have a first control unit, e.g., main microcontroller unit (MCU) that is configured to implement a charge management algorithm through a current generator and an IE voltage output. The system can also include a second MCU, e.g., a watchdog MCU, also referred to herein as a monitoring MCU, that can have an independent charge management algorithm that can monitor the main MCU and can shut the system down in the event of a discrepancy. The main and watchdog MCUs can be configured to monitor the electrodes and system in a variety of ways which can include, for example, any number of: (1) monitoring electrode voltages to protect against electrode degradation and failures and electronic failures; (2) IE current monitoring to protect against device failures in AC or DC modes; and/or (3) voltage waveform morphology analysis to protect against device failures. During AC mode, blocking capacitors can be switched in-line. The main and watchdog MCU can cross-check each other for proper orientation. The system can include a third MCU, e.g., a supervisor watchdog MCU that can prevent devices from being reset. In external non-implanted variants, only a clinician may be allowed to change batteries to avoid stim cycling termination when the electrodes are loaded with charge.

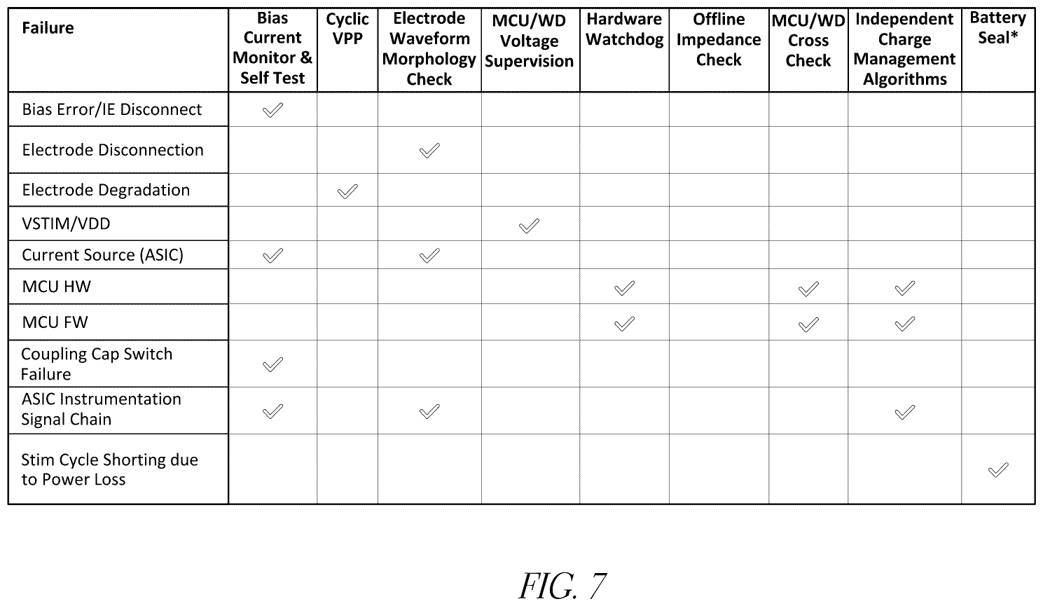

FIG. 7 shows a table of non-limiting potential failure mechanisms listed in rows and mitigation mechanisms in columns. A check mark indicates which mitigations protect against which failures, according to some embodiments. When failures occur, stimulation is stopped either immediately (instant off) or at the end of a stimulation cycle when finishing in a charged balance state is beneficial. For example, bias current monitor out of range protects from a surface electrode (IE) disconnection, a current source error, a coupling capacitor error, or instrumentation signal chain error resulting in instant off stimulation. Cyclic VPP out of range (e.g., 10 or other numbers of cycles) is a primary mechanism protecting from long term electrode degradation resulting in the stimulation being ended at the completion of a stim cycle. Waveform morphology violation (e.g., 10 or other numbers of cycles) protects against electrode disconnection, current source failure or an instrumentation error resulting in the stimulation cycle being ended and the completion of a stim cycle. MCU/WD voltage supervision protects against stimulation or other power supply issues and terminates stimulation immediately and possible power off. Hardware watchdog protection protects against firmware/MCU failures resulting in general reset and ending the stim cycle immediately. Offline impedance check—precheck to exclude failed electrode and insufficient electrode capacity. MCU/WD cross check assures that both MCUs are operating properly resulting in general reset and ending the stim cycle immediately. Independent charge management algorithms (two different algorithms with independent code bases) protect against firmware bugs and unanticipated algorithm deficiencies, resulting in general reset and ending the stim cycle immediately.

In some embodiments, a device can include a virtual ground configured to be operably connected to the indifferent electrode where the virtual ground can be set to any level to minimize power dissipation.

In some embodiments, current used from the output multiplexers are measured to detect any failures in the active silicon components that are tied directly to the body to prevent unintended DC currents due to part failures that occur especially due to ESD discharge damage.

In some embodiments, a device comprises any number of the following mitigation mechanisms: (a) indifferent electrode current monitoring halts operation if the bias currents deviate from a preset minimum and maximum range, the current used can be processed with a statistical process to remove noise; (b) Electrode Voltage monitoring either from each working electrode to the indifferent electrode, each working electrode to a reference electrode, or between a pair of working electrodes; (c) Electrode monitoring is resolved either instantaneously or statistically across a preset time from, for example, 1 μs to 1 hour or more or less, or synchronized to waveform transitions, statistics can include: mean, median, variance, minimum, and/or maximum; (d) Electrode monitoring can examine the electrode voltages in their entirety or break it into components using either a filter mechanism or by subtracting out components based upon what is known about the electrode, e.g., what is measured or the specifications of the electrode. As one example, the aforementioned above filtered voltage—stimulation current * measured access resistance can be below a specified value.

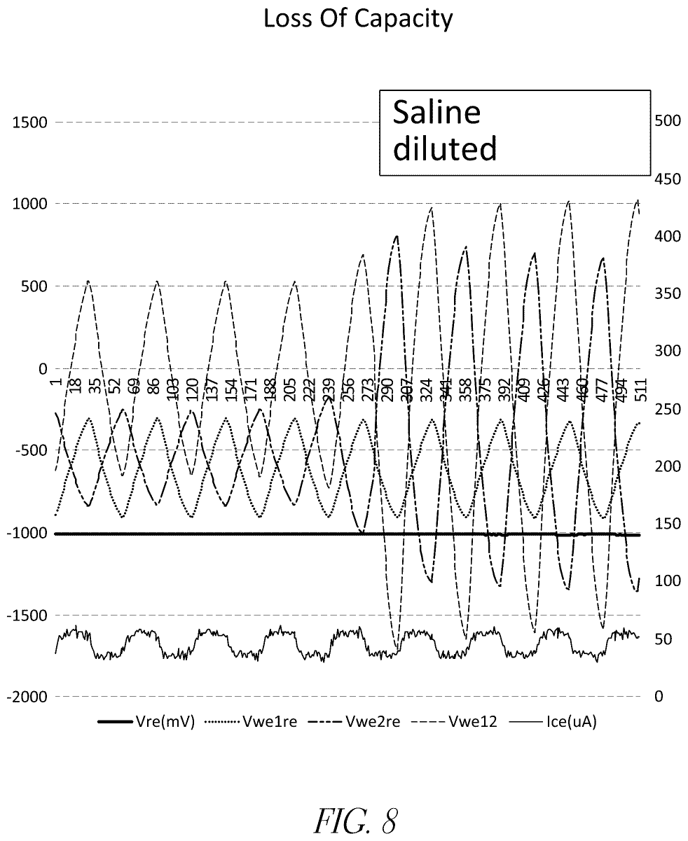

FIG. 8 illustrates a graph relating to lost capacity—which can be related to voltage protections. Cyclic VPP: VPP−2*RA*I, which can help ensure that peak voltage over a stimulation cycle stay within prescribed limits to assure an electrode has sufficient capacity over time. Electrode waveform morphology (sawtooth) can help ensure that the electrode voltage waveform is as expected for prescribed currents—which can help assure that the system is operating properly.

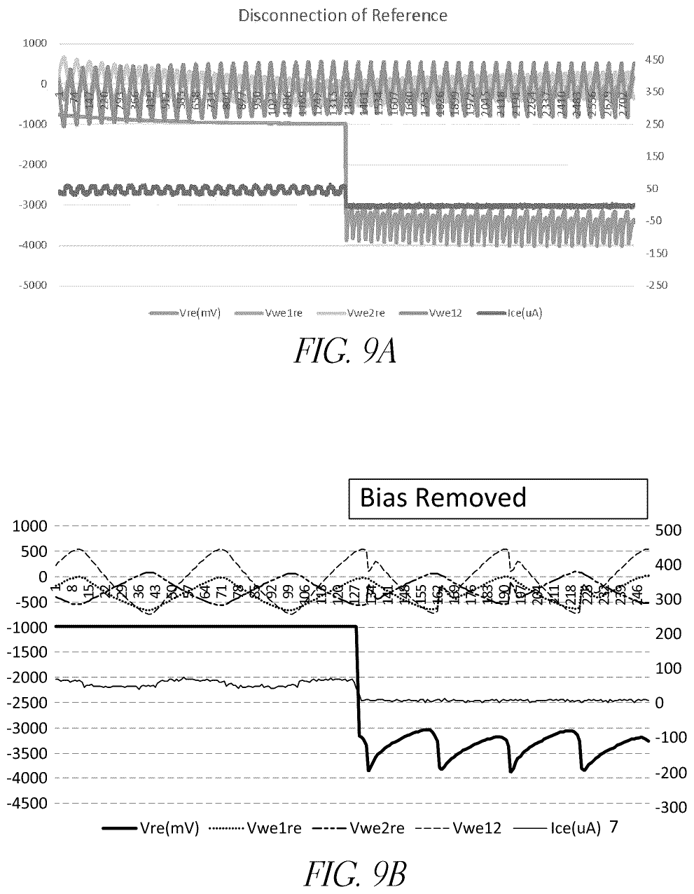

FIGS. 9A and 9B relate to bias current monitoring. FIG. 9A illustrates a graph relating to the disconnection of a reference electrode. FIG. 9B illustrates a graph relating to the bias current being removed. Bias current monitoring can protect against various faults, which can include IE failure (open circuit or Hi-Z), WE failure (open circuit or Hi-Z), WE current source failure (Hi or Low), IE voltage source failure (no current of forces current source into failure), and/or capacitor bypass switch failure (current leakage or fail open). For DC specific modulation modes, the monitor can check that the bias is in the correct range (e.g., 25-75 μA). For AC specific modulation modes, the monitor can check that DC current is less than a predetermined value, such as, for example, ≤100 nA (e.g., electronic or multiple capacitor failures).

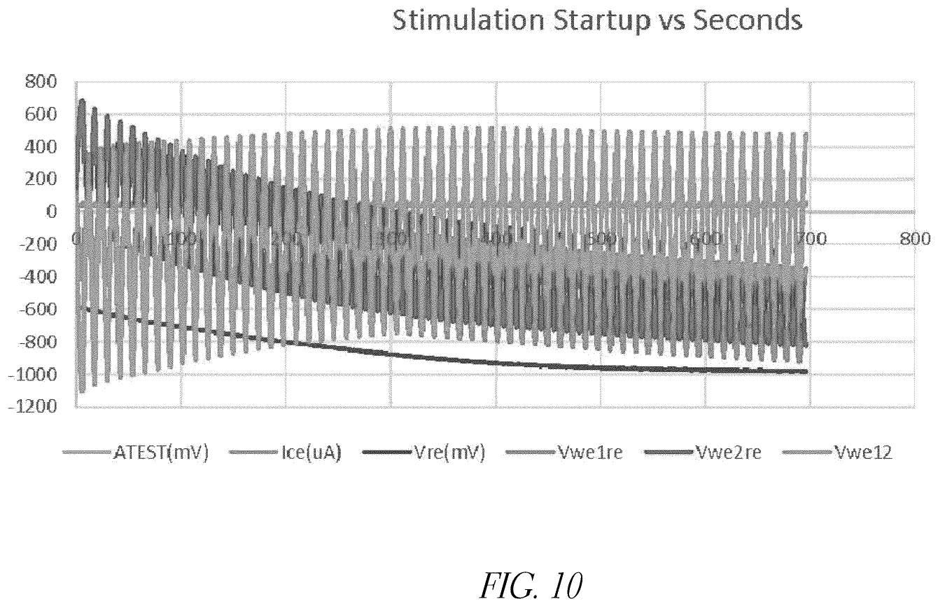

FIG. 10 relates to reducing (e.g., minimizing) irrecoverable charge. FIG. 10 illustrates stimulation startup against time in seconds. The injection of bias current can place the electrode into an operational voltage range that can allow the charge and/or electrode life to be increased, such as maximized. The operating condition of the electrode can be determined, which can include determining that the electrode is in good operating condition. It can be determined that the electrode is in good operating condition by, for example: (1) determining (e.g., assuring) that the peak of the referenced voltage waveform is below a calculated or empirically calculated voltage, and/or (2) integrating the voltage of a cycle as an indicator of irreversible charge and/or determine (e.g., assure) that it is under a specific threshold.

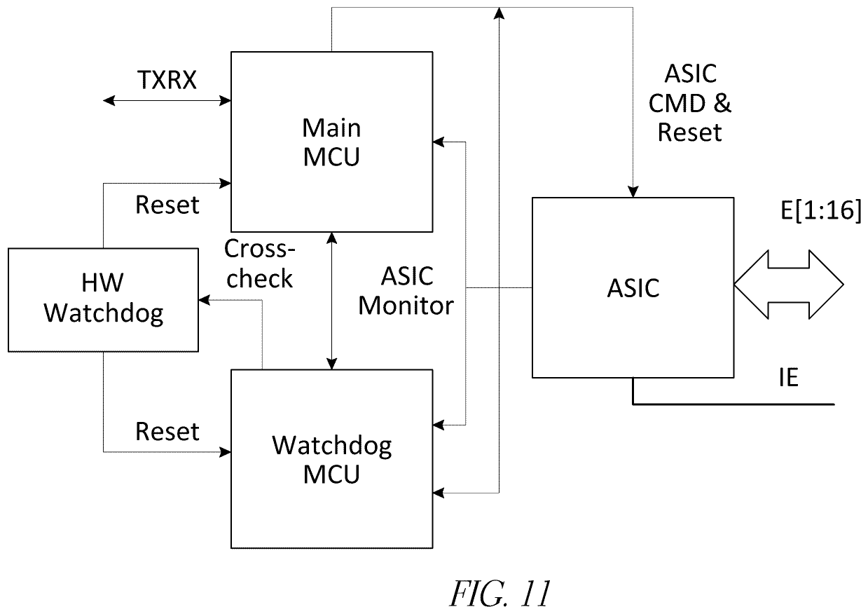

FIG. 11 illustrates an example block diagram relating to HW and FW fail-safe. Charge management algorithms—main MCU controls ASIC (or discrete current generator). MCU/WD voltage/current supervision—independent ADCs and algorithms in main and watchdog can be kept alive by Watchdog MCU assuring HW and FW are operational. MCU/WD cross check—Main and Watchdog MCU check-up on each other to assure HW and FW are operational. MCU/WD ASIC reset—either the main or watchdog MCU can reset ASIC when a problem is detected.

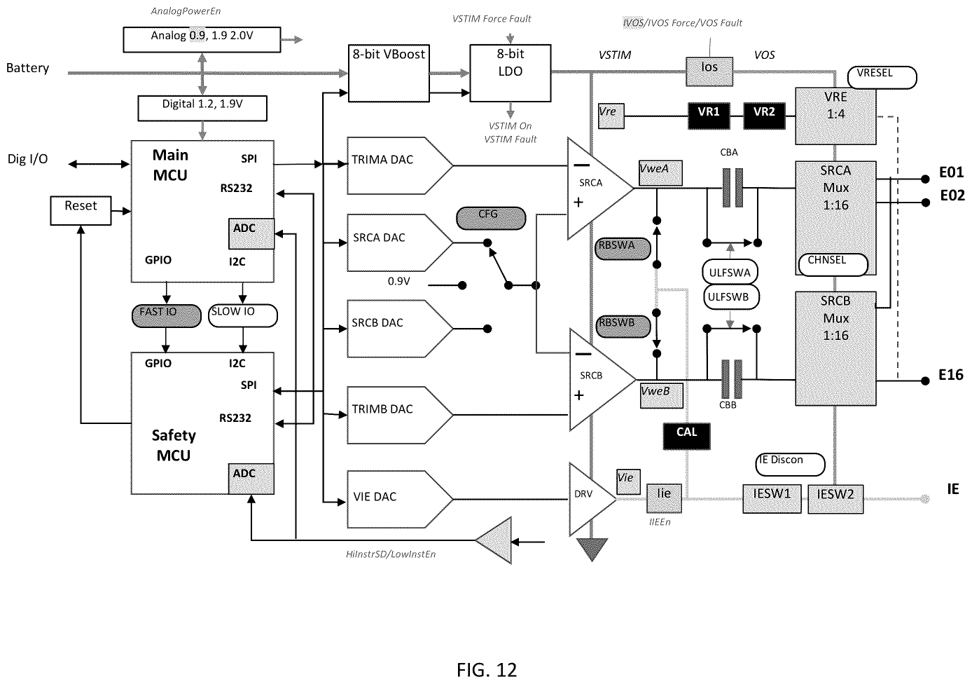

A block diagram of an example stimulation engine (sometimes referred to as a waveform generator or therapeutic waveform generator) that can provide DC and AC therapy, including simulation with a single architecture is illustrated in FIG. 12. A conventional approach for medical devices is to utilize only one microprocessor to both control generating stimulation on an electrode and to check the stimulation on the electrode. Such approaches render as therapeutic system susceptible to a double failure that can result in patient harm. From a safety standpoint, this results in all system software as having a higher risk classification (e.g., of higher risk class C instead of lower risk class A or B). By providing an independent microprocessor that monitors the electrode, such as shown in the architecture of FIG. 12, three simultaneous failures would need to occur to create a harmful situation to a patient. The independent safety microprocessor (e.g., the Safety MCU, as shown in FIG. 12) reduces overall risk and improves safety. This allows the safety classification of the software and the system to be reduced (e.g., from higher risk class C to lowest risk class A). This greatly simplifies the design and testing of the system, as well. The stimulation engine can stimulate with a single bipolar pair of current generators. The main micro controller unit (MCU, processor or controller) can generate stimulation waveforms and the safety MCU can continuously verify proper system operation. The Boost Converter and LDO (low dropout regulator) can set the low noise stimulation compliance voltage (VSTIM) to drive Bipolar Current Generators SRCA, SRCB. The current generators SRCA, SRCB can be sufficiently fast to support AC waveforms having 1. 5, 10, 15, 20, 25, 30, 35, 40, 45, 50, 55, 60, 65, 70, 75, 80, 85, 90, 95, or 100 μs rise and fall times. Driving voltage for such generators (VSTIM) is sufficiently high such that that the current generators operate within the common mode input range to preserve power.

During low duty cycle AC (e.g., tonic) stimulation, VSTIM can be substantially (e.g., mostly) set to zero volts-shutting down the stimulation engine to preserve power. Since the system can operate from a single supply, the virtual ground (VIE) can be set to the mid-rail for AC. During DC (e.g., ULF), VIE can be set to a fraction of VSTIM dependent upon the bias voltage, created by the bias current, that develops around the electrode operating voltages to save power. Stimulation voltages and currents can swing around VIE. Since bias current moves voltages below the mid-rail, the VIE can be designed to chase those voltages to save power. For DC mode, VIE can absorb the constant offset current and can be connected to the indifferent electrode (IE) which can be a surface electrode for the stimulation engine and the can (e.g., housing or enclosure) for the stimulation engine. During AC modes, the charge imbalance that can collect on the inside of blocking capacitors CBA, CBB due to charge imbalance can be discharged onto the VIE. In AC mode, the indifferent electrode IE is disconnected from the patient. VIE can be further set either toward the VSTIM or ground rail when the electrode achieves a bias voltage to further optimize power.

Bipolar current generators SRCA, SRCB can push or pull current to support the required balanced or intentionally unbalanced bipolar simulation mode. For example, the generators SRCA. SRCB can operate in bipolar mode and can be configured to deliver opposite and equal or unequal currents. In AC mode, they can be configured to generate equal and opposite currents. In DC mode, they can be opposite, and may optionally include slightly offset currents. Since the matching requirements are very high (˜1 μA) separate trim DACs (digital-to-analog converters) (TRIMA DAC, TRIMB DAC) for both currents are employed. Both DC offsets and AC zeroing can be accomplished by utilizing the trim DACs, e.g., TRIMA DAC, TRIMB DAC. For example, residual post-calibration non-linearities may exist due to a mismatch between the complementary pair of current generators. To address, this one of the current generators (e.g., SRCA) may be arbitrarily selected as a reference source. A secondary calibration may be performed to force the adaptive current generator (e.g., SRCB), to match the reference source (e.g., SRCA). This calibration is applied to the TRIM DAC (e.g., TRIMB DAC) on the adaptive current generator (e.g., SRCB), which then matches the differential non-linearities, and allows the normal calibration to correct the overall non-linearities (including the differential non-linearities). In DC mode, source DACs SRCA DAC, SRCB DAC can deliver the slowly varying current and, as mentioned herein, the trim DACs can trim the values and set the offset. The source DACs SRCA DAC, SRCB DAC can be updated by software through the SPI port when the current value needs to change, but can change at any rate, up to 100 Hz or more, or others, but during stimulation plateaus, a single stimulation value can persist for several seconds.

In AC mode, stimulation can vary quickly so the cathodic (activation) current amplitude can be programmed into one source DAC, e.g., SRCA DAC and the recovery amplitude can be programmed into the other source DAC, e.g., SRCB DAC before the start of stimulation or when stimulation is changed. AC pulses can be formed quickly and with high efficiency by flipping the source of each current source to be either one source DAC, e.g., SRCA DAC, mid-supply (for zero current), or the other source DAC, e.g., SRCB DAC.

Two blocking capacitors CBA, CBB can be used to guarantee DC blocking in AC mode but can be bypassed in DC Mode. Rebalances switches RBSWA, RBSWB (also referred to as DSW1, DSW2, REBALA, REBALB, D1, D2) can recover charge from the blocking capacitors CBA, CBB and can be used for self-test and other calibration modes. Discharge switches RBSWA, RBSWB may be used to discharge blocking capacitors CBA, CBB. In one embodiment, the stimulation engine includes one or more switches, e.g., ULFSW that can close to bypass/short blocking capacitors CBA, CBB when operating in DC (or ULF) mode, but open to cause the driving current to pass through the blocking capacitors CBA and CBB when operating in AC mode. The driving current is then directed through multiplexers SRCA MUX, SRCB MUX and routed to a desired electrode E01-E16. During AC mode, the capacitors CBA, CBB may be used to assure that the current to the electrodes E01-E16 is balanced.

In one embodiment, blocking capacitors CBA, CBB are located on the input side of the multiplexers SRCA MUX, SRCB MUX. Such arrangement avoids the need to place a separate capacitor on each electrode E01-E16, which simplifies circuit design and reduces the footprint of the implantable stimulation engine. The multiplexers SRCA MUX, SRCB MUX advantageously allow significant customizability of the stimulation engine. For example, an implantable lead of the simulation engine may include 16 electrodes. The multiplexers SRCA MUX, SRCB MUX allow the stimulation engine to be configured to deliver any desired electrical waveform to any desired electrode. Furthermore, the multiplexers SRCA MUX, SRCB MUX or other multiplexer, e.g., VRE, allows any of the electrodes to be selected to function as a reference electrode. With such configurability, the can, or indifferent electrode of the stimulation engine does not necessarily need to be utilized as the stimulation engine's reference electrode. Instead, any one of the electrodes E01-E16 may be utilized as a reference electrode. Furthermore, each electrode may be selected by a multiplexer VRE, SRCA MUX, SRCB MUX, to operate as an anode or cathode of the stimulation engine's tissue stimulation signal.

Also, during AC mode, the IE can be disconnected through a fault tolerant set of series IE switches IESW1, IESW2. The IE current sensor lie can be used for fault detection and to measure a constant offset current in DC and can also be used for self-test calibration and diagnostic modes. Current steering diodes (not shown) can protect all system outputs against over-voltage on the terminals and the series configuration can protect shorting to ground from single point failures.

Electrocautery can present a significant challenge to therapeutic waveform generators, including SCS devices because it can source 200 W @ 1000 V @ 490 KHz. Defibrillators present a similar challenge. These voltages can result in damage to the driving electronics, resulting in device malfunction and failure. This challenge is amplified in devices with distantly spaced electrodes, such as those with spacing between the working and indifferent, or return, electrodes. The indifferent and working electrodes can be separated by a large distance, resulting in a high potential difference between electrodes that is fed into the electronics. An example of such a configuration is one that that utilizes an active can, where the metal casing is an active component in the electrical circuit, where the can is not placed near the working electrodes. Another example includes configurations with inactive cans, where the can is not an active component in the electrical circuit, and an alternate indifferent or return electrode, including but not limited to a surface electrode, separate implanted electrode, or electrode contact on an electrode array, where the indifferent or return electrode is not near the working electrodes. Another example includes configurations with locally placed indifferent, or return, electrodes, including but not limited to configurations in which an electrode contact on an array is specified as a return or indifferent electrode. In all of these non-limiting examples, the spacing between working electrodes typically presents less risk due to the comparatively reduced separation distance to an indifferent, or return, electrode. However, these working electrodes present a non-zero risk for electrocautery damage.

It is therefore desirable to include protection mechanisms to safeguard against electrocautery, defibrillator, or other high-voltage/high-current damage. Some therapeutic waveform generators and SCS systems utilize capacitors at the electrodes to provide protection against electrocautery damage. However, the requisite lack of isolation capacitors in low-frequency, ultra-low frequency, and DC systems at the electrodes requires an alternate approach that does not rely upon capacitors.

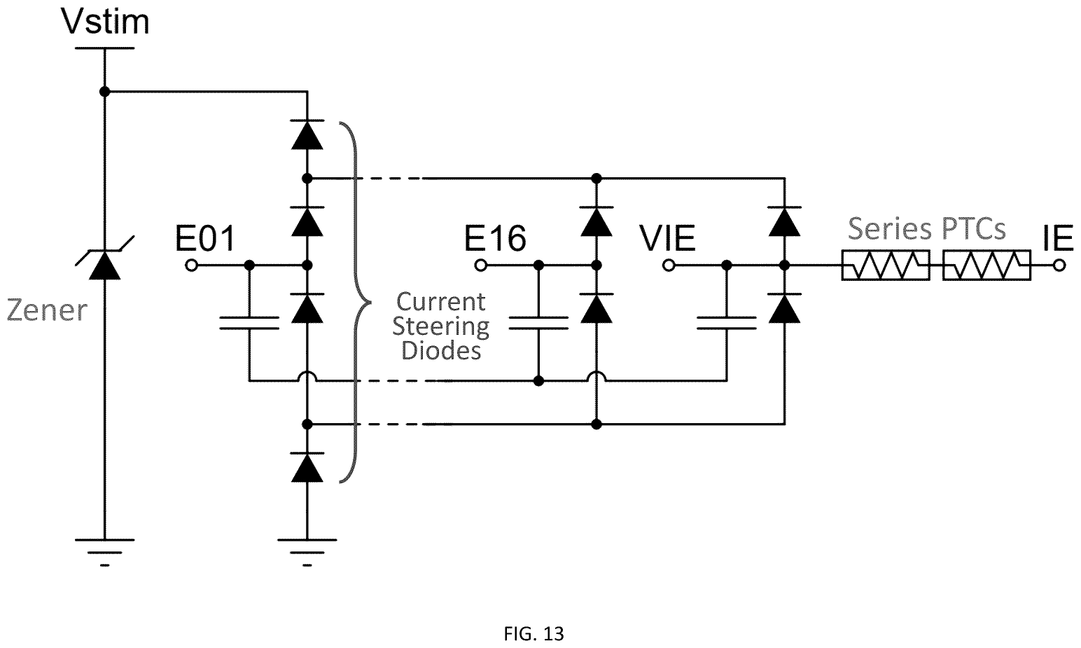

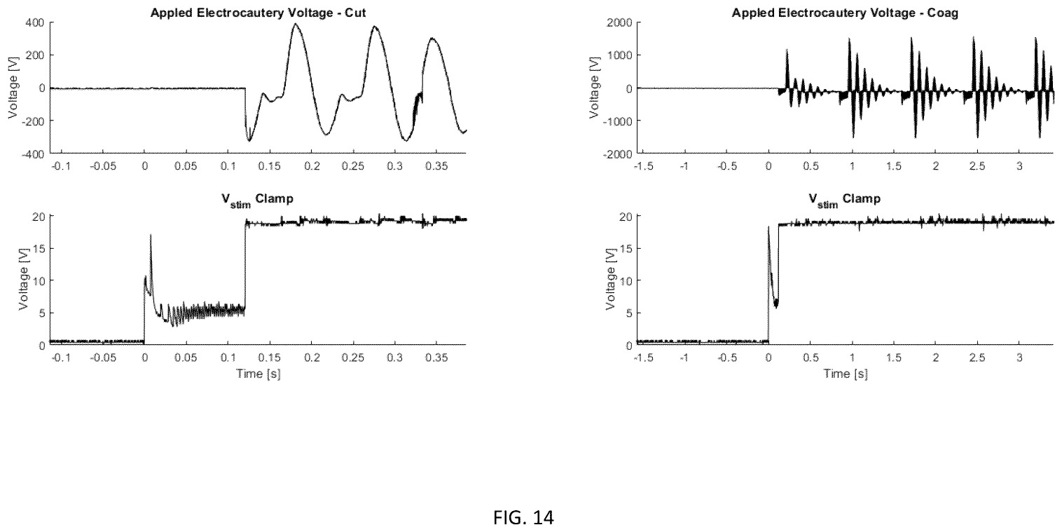

FIG. 13 illustrates one example of a system for protecting against high voltage and/or high current that utilizes current steering diodes and fast-acting positive temperature coefficient (PTC) devices. A Zener diode in series with two PTCs connects an electrode can to the driving electronics. In the presence of externally applied voltages of moderate magnitude, such as electrostatic discharge, the Zener diode clamps the external voltage to a safe level, such as 5, 10, 15, 20, 25, less than 25, or less than 30 V. Externally applied voltages that result from electrocautery present high voltages that would result in irreversible breakdown of the Zener diode. Placing one or more PTC devices in series with the Zener diode safeguards the Zener diode from damage to disconnect the electrode can from the driving circuitry. High voltage/current will result in a temperature increase across the PTC, increase in PTC resistance, and a significant reduction in the power burden of the Zener. FIG. 14 illustrates how this series configuration can enable voltage clamping to ˜18 V in the presence of high-voltage electrocautery signals.

Referring back to FIG. 12, instrumentation of the stimulation engine can include measurement of the working electrode voltages VweA, VweB, the indifferent electrode voltages Vie, and the reference electrode voltage Vre where the reference electrode is 1 of the total number, e.g., 4, 16, etc., of selectable working electrodes. All of the foregoing instrumentation can be used for diagnostics, calibration, and/or to implement safety and control measures. All voltages can be buffered before routing into both the main and safety MCU ADCs where these independent MCUs can be used, at least in part, to achieve a safe fault tolerant system.

Four (or more or less) (e.g., E01, E08, E09, E16) of the sixteen (or more or less) working electrodes can be hardwired to the reference electrode multiplexer VRE. One (or more or less) of those electrodes can be selected electronically as a reference and can be placed into electrical communication with a VREF Amplifier (not shown) though two series resistors VR1, VR2 to limit the current to, e.g., <5, 10, 15, 20, 25, 30, 35, 40, 45, or 50 μA in the case of a worst-case amplifier failure. Reference electrode multiplexer VRE can be selected momentarily when sampling the reference voltage. A valid reference voltage Vre measurement can be made by any electrode that is not being used to stimulate or otherwise provide a therapeutic signal to a patient.

AC neurostimulation systems can rely primarily on isolating active circuitry from the body with series capacitors. In conjunction with internal discharge resistors/switches the capacitors not only protect from circuit failures but provide charge balanced waveforms. High-capacity electrode systems that utilize balanced or imbalanced charge biphasic waveforms that operate at ultra-low frequencies utilize DC stimulation and cannot readily utilize capacitors. As a result, alternate safety mechanisms must be implemented. Similarly, the same limitations may apply for systems that use balanced charge biphasic waveforms (e.g., ULF waveforms on balanced systems). Therefore, alternative (non-capacitor-based) solutions may be utilized in such systems, as well.

In one embodiment, DC mode operates by providing an imbalanced charge ultra-low frequency imbalanced waveform that can operate the electrode within its protective voltage region where long term electrode capacity is optimized and preserved. The safety mechanisms can assure that resulting electrode voltages stay within the prescribed range as evaluated by at least two independent mechanisms, even in the case of one or more system fault, any detected faults can result in stimulation shutdown and power down of the stimulation engine.

To better understand such safety mechanisms, the electrode can be modelled by a simplified Randles Cell: a series access resistance (Ra) and capacitance (Cdl) and polarization resistor (Rp or Rct). The polarization resistor because it is about >10× larger than Ra will be ignored in this treatment. The total voltage across the electrode (Vt) is equal to Ra*I+Cyclic Vpp where Cyclic Vpp is the peak-to-peak voltage across the capacitive component (Cdl) of the electrode. Given this relationship, Va (from Ra×I) and Cyclic Vpp can be separated on each stimulation cycle using real-time measure of Vt and being able to calculate Ra.

To ensure tissue safety, operating electrodes within their electrode capacities can be important. Driving electrodes outside of their capacity eventually may reduce electrode capacity and facilitate reactions that may impact tissue health and cause irreversible electrochemical reactions. Cyclic Vpp is the primary measure of electrode health and is inversely proportional to the capacity of electrodes. Cyclic Vpp is expected to be fairly constant once the electrode has achieved steady state operation. If changes to the electrode over its life occur, these can be detected via Cyclic Vpp and stimulation can be adjusted to ensure operation within the electrode capacity, or the stimulation electrodes may be changed as needed.

In AC stimulation mode, blocking capacitors CBA, CBB can be positioned between the electrode contacts and the current outputs to protect against DC. In DC neuromodulation mode, e.g., stimulation mode, the capacitors CBA, CBB can be disabled with a switch ULFSW. A variety of failure modes may be realized with capacitors and switches. For example, there can be a failure mode such that if a capacitor CBA, CBB fails, it can pass DC to the indifferent electrode IE, and the DC current can be detected (e.g., by current sensor lie, etc.). There can be a failure mode such that if a capacitor fails, the other capacitors can protect the body from DC through the other electrodes. There can be a failure mode such that if a switch fails, DC can pass to the indifferent electrode that can be detected. There can be a failure mode such that if the current outputs in AC mode fail, capacitors can protect the body from DC. There can be a failure mode such that if the current outputs in DC mode fail that can be detected on the indifferent electrode.

The stimulation engine may include two processors: a Main MCU and a watchdog or Safety MCU. In DC mode, the Main MCU implements a charge management algorithm (CMA) using a current generator and an IE voltage output (virtual ground). The watchdog or Safety MCU can have an independent charge management algorithm that monitors the Main MCU and can shut the system down in the case of a discrepancy. The Main and Safety MCUs can monitor each other with an independent ADCs monitor: electrode voltages to protect against electrode degradation and failures and electronic failures; IE currents to protect against device failures and in appropriate DC levels; and/or voltage waveform morphology to protect against device failures. The Main and Safety MCUs can cross-check each other for proper operation. The Main and Safety MCUs can both reset/disable the ASIC. There can be a variety of failure modes. For example, if the charge management algorithm fails in the Main MCU, the Main MCU may observe electrode voltage issues; the Main MCU may determine if IE current goes out of range; the Safety MCU may observe electrode voltage issues; and/or the Safety MCU may determine if IE current goes out of range. If the Main MCU malfunctions, the Safety MCU can observe such condition and request stimulation stop and then reset the Main MCU and ASIC and/or the supervisor chip can observe condition and force both Main and Safety MCU and ASIC into reset. If the Safety MCU malfunctions, the Main MCU can observe conditions and request stimulation stop then reset the Safety MCU and ASIC and/or the supervisor chip can observe condition and force both the main and Safety MCU and ASIC into reset.

In addition, the Main MCU can control the current generator (e.g., using TRIMA DAC & SRCA DAC that controls current source SRCA, SRCB DAC& TRIMB DAC that controls current source SRCB and an IE voltage output (virtual ground) (e.g., using VIE DAC and amplifier DRV). The Safety MCU can have an independent charge management algorithm that can monitor the Main MCU and can shut the system down in the case of a discrepancy. The Main and Safety MCUs monitor each other, and each can include an independent ADC monitor: IE currents to protect against device failures and inappropriate DC levels. The Main and Safety MCUs can cross-check each other for proper operation. The Main and Safety MCUs can both reset/disable the ASIC. There can be a number of failure modes, which can include that capacitor (e.g., CBA, CBB) or capacitor bypass switch (e.g., ULFSW) failure can cause IE current flows.

The stimulation engine (e.g., the stimulation engine of FIG. 12 or other stimulation engine as described herein) may be implemented as an application specific integrated circuit (ASIC). As such, the stimulation engine could also include more than two bypassable blocking capacitors, e.g., one blocking capacitor for each electrode, e.g., 16 capacitors if 16 electrodes were employed. The current output the ASIC can be clocked by an oscillator; receive and execute commands from the main MCU; and/or analog differential electrode voltages buffered by internal and then external amplifier. There can be various failure modes. If the current output ASIC current source, oscillator, and/or amplifier fail, the Main and Safety MCU can detect errors in IE currents and/or the Main and Safety MCU can detect errors in electrode voltages including: cyclic VPP, waveform morphology, and/or electrode voltage.

In one embodiment, an IE amplifier DRV of FIG. 14 can buffer a voltage generated from VIE DAC commanded from an MCU to drive the indifferent electrode IE, which is sometimes referred to as a virtual ground. In DC mode, the IE can absorb a bias current that protects the electrode. In AC mode, the IE current typically not utilized. The current sensor lie can measure the current to the IE to test for various failure modes. For example, if the IE amplifier DRV fails, the current output may not accept additional current from the IE, causing the electrode voltages to error and be noted by the MCUs. In DC mode, if the current sensor lie fails out of range, the MCUs can shut down stimulation. During AC mode, the current sensor lie can be periodically tested to avoid the double fault case when the AC current sensor lie fails low and a capacitor switch e.g., ULFSW fails.

The stimulation engine systems disclosed herein can differ from other systems in various aspects. In one aspect, the systems disclosed herein can use an ultra-low frequency (ULF) stimulation waveform and a low current offset (“current offset”), which may be in the form of a constant current offset. The ULF waveform current amplitude can be adjusted for the individual patient to achieve efficacy. The low current offset can be used to bias the operating voltage of the working electrodes such that they operate at an increased (e.g., maximum) long-term charge transfer potential.

ULF waveforms can be charge balanced over a stimulation cycle (minus the current offset) and preferably are stopped on the end of a cycle to avoid adverse patient perceptions as well as undesired, unperceived neuromodulation. Likewise, in some cases the ULF waveform has all smooth transitions (e.g., rounded edges, not a square wave) between waveform segments to minimize such adverse patient perceptions and undesired, unperceived neuromodulation. In a bipolar mode, a current offset can be introduced into the system by shifting the charge-balanced ULF waveforms each by a predetermined fraction, e.g., ½ the target current offset level, and the unbalanced portion of the waveform, the current offset, can removed through the indifferent electrode (IE).

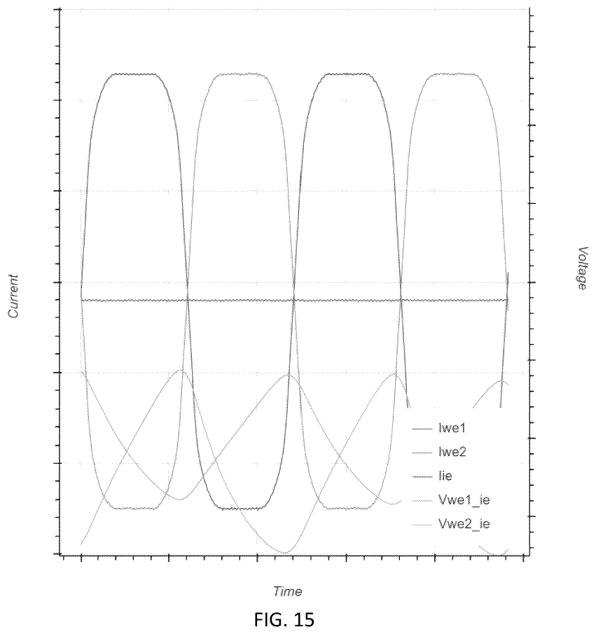

A non-limiting example measurement of a DC (e.g., ULF) waveform stimulating a DC lead in-vitro (e.g., generated by the stimulation engine, and during the simulation engine's DC mode of operation) is shown in FIG. 15. Bipolar currents are generated by sourcing current from SRCA and sinking current SRCB, and then alternatively sourcing from SRCB and sinking through SRCA to switch the polarity. Switching the polarity at a specific rate allows the generation of any of the frequencies or frequency ranges described herein with a stimulation amplitude of a magnitude of any stimulation amplitude described herein. In one embodiment, the bias of each electrode is any bias value described herein, and may be in the mA, μA or nA range. Bias is generated by intentionally mismatching the source and sink currents between the current generators and may be set in a particular polarity without a frequency. If the bias current is constant and negative, then that may shift magnitude of the positive value of the ultra low frequency waveform. If the bias current is constant and positive, that may shift the magnitude of the negative value of the ultra low frequency waveform. A complex non-DC bias can also be introduced using this technique.

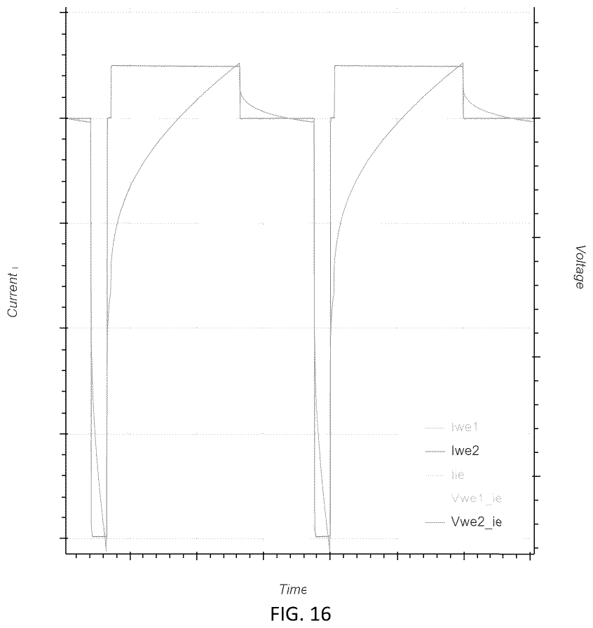

A non-limiting example of an AC waveform (e.g., generated by the stimulation engine and during the stimulation engine's AC mode of operation) is shown in FIG. 16. Only the Iwe2 and Vwe2-Vie voltages of the bipolar pair SRCA, SRCB are shown for clarity. The amplitude of the waveform is any amplitude described herein, and may be in 100, 500, 1000, 2000, 3000, 4000, 5000, 6000, 7000, 8000, 9000, or 10000 μA or any other amplitude and the recovery pulse is a fraction of the amplitude, such as ½, ⅓, ¼, ⅕, ⅙, 1/7, ⅛, 1/9, 1/10 or any other fraction of the amplitude. The recovery pulse is 1×, 2×, 3×, 4×, 5×, 6×, 7×, 8×, 9×, 10× or other multiple longer than the active pulse. The stimulation engine can drive a zero current between the activation and recovery pulse. After the recovery pulse is complete, the current sources maintain a near zero current for the remainder of the pulse for the time it takes to reach the period corresponding to the pulse frequency.

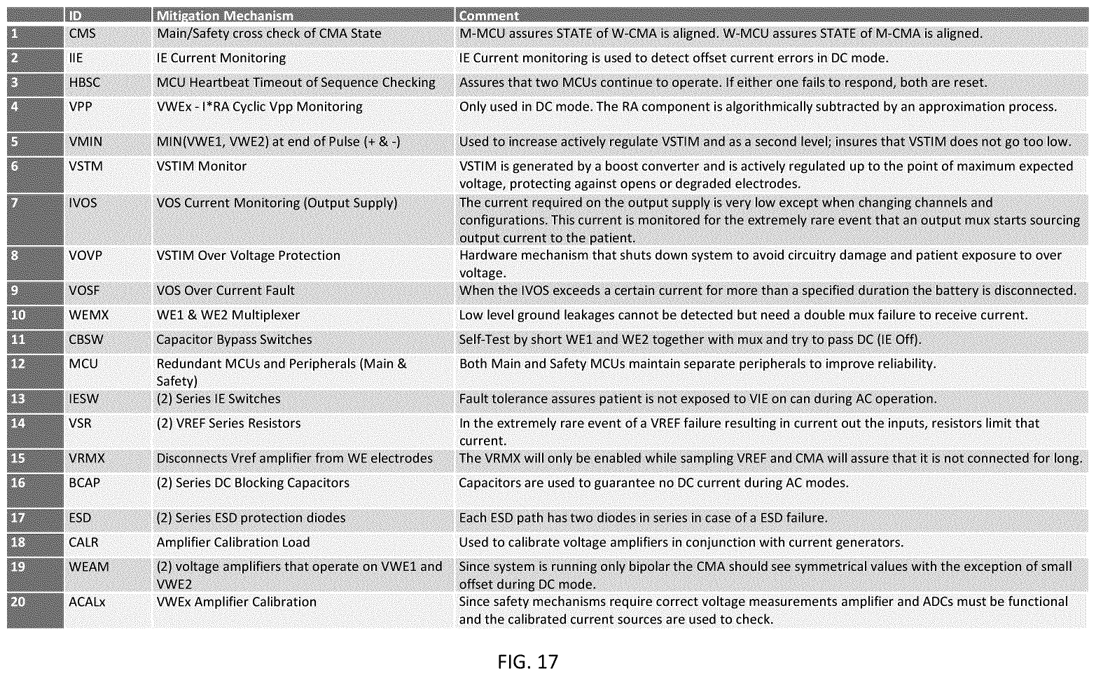

The stimulation engine can include several mitigation mechanisms and can be categorized into firmware-based Charge Management Algorithm (CMA) Components that are firmware based and Hardware Mitigation Mechanisms—as summarized in the tables illustrated in FIGS. 7 and 17. Although many of the mitigation mechanisms are described with respect to systems that deliver a ULF current with a bias current, the same mitigation mechanisms may be used with systems that deliver a ULF current without a bias current, as well.

CMA Components can be implemented independently on independent multiple, e.g., Main and Safety MCUs. Two independent firmware images running two independent algorithms running on two independent processors can have a very low probability of failing within a finite time window.

The table in FIG. 17 lists several mitigation mechanisms or fault detection events. Each row in the table corresponds to a mitigation mechanism. The columns in the table correspond to a mitigation mechanism ID, a description of the mitigation mechanism, and comment relating to the mitigation mechanism. Any number (e.g., all or a subset) of mitigation elements disclosed herein can be included and/or excluded in neuromodulation engines depending on the desired clinical result. Furthermore, any one of the mitigation mechanisms, when detected by a controller or processor, can cause a system response, including, but not limited to, preventing operation of the stimulation engine, removing the Vstim signal, disconnecting the battery, etc. In addition, the mitigation mechanisms or fault conditions may be detected by hardware, software, and/or firmware of the stimulation engine.

The stimulation engine may employ a variety of safety mechanisms to assure proper function and redundancy checking of system performance. For example, the CMS mechanism relates to providing two or more processors (e.g., Main MCU (M-MCU), Safety MCU (sometimes referred to as the Watchdog MCU or W-MCU)) that independently operate and monitor the performance of each other, including the operation of the other processor's charge management algorithm (CMA). Two processors also provide redundancy so the stimulation engine may continue operating upon the unlikely failure of one of the processors.

The IIE mechanism refers to providing current monitoring to detect current errors when operating in DC mode. IIE current errors may result from either a SRCA, SRCB, VIE failure, an interconnection error, or a short to supply or ground For example, the stimulation engine of FIG. 12 may include a current sensor lie, as described in greater detail herein.

The HBSC mechanism refers to monitoring an MCU heartbeat signal and sequence checking. If either the Main MCU or the Safety MCU fails to generate a heartbeat signal, or if its sequence includes an error, one or both MCUs are reset. A heartbeat signal indicates that the microprocessors (Main MCU and Safety MCU) are operating. However, even if operating, it is still possible for them to be desynchronized. A heartbeat count provides an additional safety measure to assure that both processors are running extended periods of time without undetected resets and to assure that they stay synchronized. Such a configuration may be used to detect unexpected independent MCU reset conditions (e.g., desynchronization, etc.).

A VMIN monitor can indicate whether the VSTIM (stimulating Voltage) (e.g., as detected at a selected working electrode E01-E16 ) is at a desired level. A VMIN signal can indicate whether VSTIM exceeds or fails to achieve the desired level, or whether it is outside of a threshold difference with a desired level.

The VSTM mechanism refers to a Vstim or stimulating voltage monitor. Vstim may be generated by a boost converter, as shown in FIG. 12. Vstim may be actively regulated and monitored to assure that it does not exceed a maximum expected voltage. This protects against open circuits or degraded electrodes.

A VOSF monitor can indicate whether an over-current fault has occurred. For example, when the IVOS signal exceeds a threshold current level for more than a specified duration, a fault condition may be detected, and a system response (such as those described herein) may occur. The IVOS signal can correspond to a monitored current signal from the output power supply. The power supply current to a multiplexer is monitored, as a current exceeding an expected value can indicated multiplexer failure.

A VOVP mechanism refers to a VSTIM over voltage protection mechanism. This hardware mechanism shuts down the system to avoid circuitry damage and to avoid exposing the patient to undesired voltage levels.

The VPP mechanism refers to monitoring electrode health, and is described above with respect to FIG. 8. Other mechanisms to monitor electrode health and performance may be provided, as well. For example, real time monitoring of electrode performance by determining each electrode's series access resistance (Ra), capacitance (Cdl) and polarization resistance (Rp or Rct) can indicate whether the electrodes are operating as anticipated. For example, a change in electrode structure, plating, and/or position within the subject can lead to a change in one or more of such electrode parameters. Real time monitoring provides an indication when an undesirable change has occurred so that system operation may be suspended, or an electrode may be identified as possibly faulty. A faulty electrode may then be removed from the stimulation engine's output path by controlling a multiplexer to avoid selecting such electrode for stimulation output.

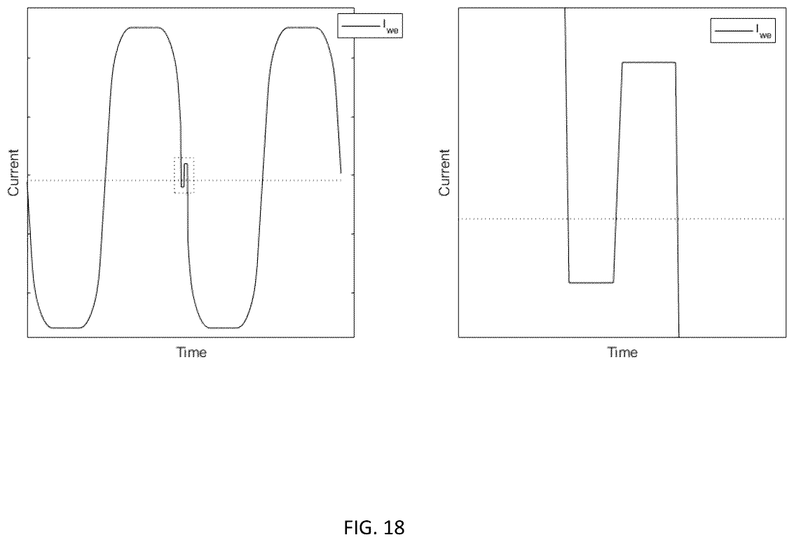

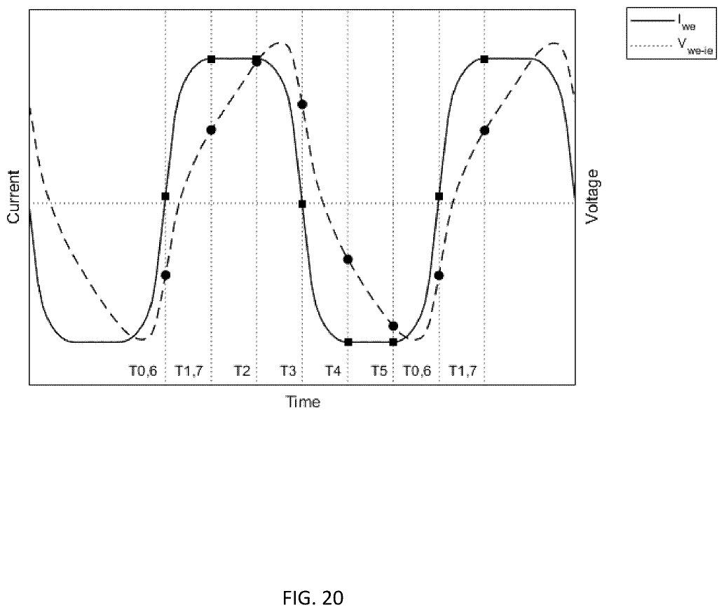

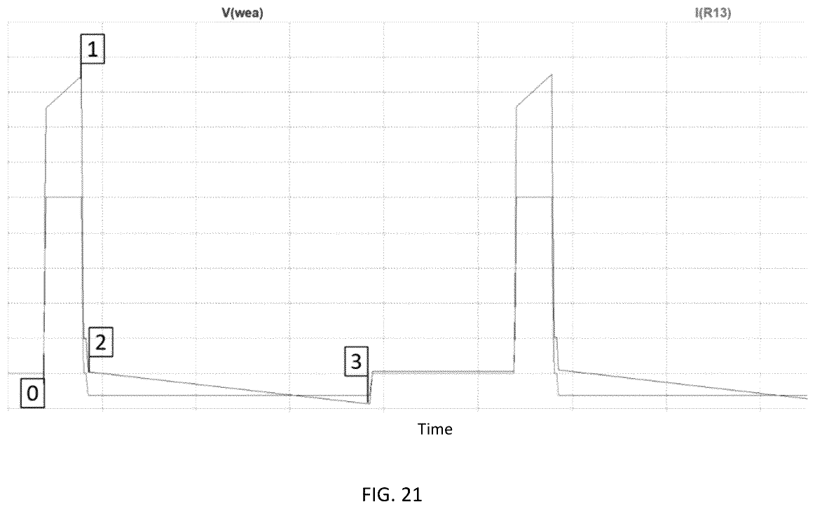

Examples of real-time measurement and calculation of circuit parameters are shown in FIGS. 18-21.

In some configurations, the series access resistance, Ra, and double layer capacitance, Cdl, can be measured using a test rectangular biphasic current pulse and potential measured between the working and indifferent electrode [Vwe-it(t)]. Rising and falling edge of the rectangular current waveform will correspond to voltage step, Vₛₜₑₚ, in the measured potential as a result of the ohmic drop across the solution resistance (Ra), with negligible contribution from the electrode capacitance to the high-pass nature of the capacitive interface. Therefore, the voltage step produced by the current step of known magnitude I will give Ra from Ra=Vₛₜₑₚ/I at the beginning and end of the biphasic pulse, and Ra=abs(Vₛₜₑₚ/(2*I)) from during the polarity reversal. The capacitance can be determined from the voltage increase, Vₚₗₐₜₑₐᵤ, from the rising to the falling edge time, tₚₗₐₜₑₐᵤ, of constant current, I, due to the approximately linear relationship between stored charge and voltage across the electrode interface. The voltage measurement will give Cdl from Cdl=I*tₚₗₐₜₑₐᵤ/Vₚₗₐₜₑₐᵤ, or for some portion of the plateau. In some configurations, this biphasic waveform can consist of a single biphasic pulse, or a series of biphasic pulses. In some configurations, this biphasic waveform can be applied prior to or at the cessation of delivery of the therapy waveform to establish initial and final values for Ra and Cdl. In some configurations, this biphasic waveform can be applied periodically during a pause in the delivery of the therapy waveform. In some configurations, this biphasic waveform can be applied between periods of the therapy waveform without pausing therapy using a very low current I and short period and subsequently small tₚₗₐₜₑₐᵤ to enable real-time assessment of electrode parameters (FIG. 18).

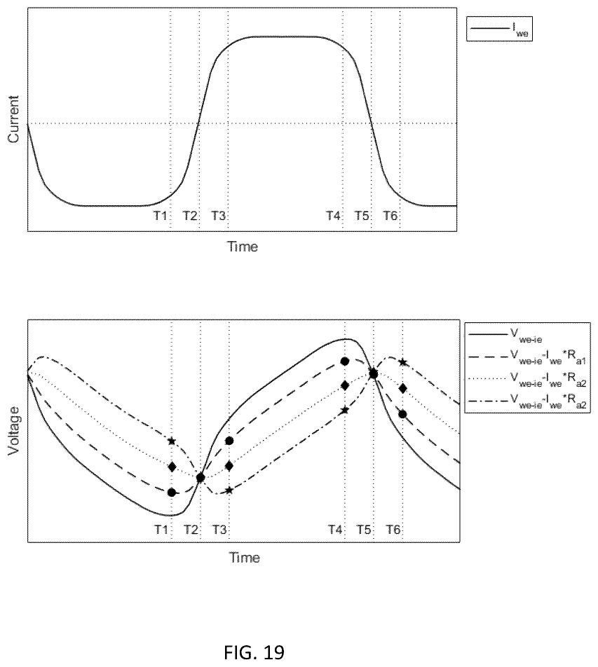

In some configurations, the series access resistance Ra and cyclic peak-to-peak voltage Vpp are approximated using a set of points sampled from the potential measured between the working and indifferent electrode [V_{we-ie}(t)] and the instantaneous stimulus current [I(t)]. Sampled points are then analyzed using an algorithm to calculate Ra and Vpp. Similar analysis can be done between the working and reference electrodes [V_{we-re}(t)]. An example of such a configuration is shown in FIG. 19.

In one configuration, the algorithm includes the following steps:
1. A time point, T₂ is set at a current zero-crossing point.
2. A set of time points is bounded by time points T₁ and T₃, where T₁=T₂−δ_{T} and T₃=T₃+δ_{T}, where δ_{T}>0.
3. V_{we-ie}(t) is sampled at all time points bounded by T₁ and T₃
4. I(t) is sampled at all time points bounded by T₃ and T₃ from the device commands to output, or sampled from an output current monitor
5. A set of values for access resistance, Rₑₛₜ, is constructed from entries Rₙ for Rₘₐₓ≥Rₙ≥Rₘᵢₙ, where Rₘᵢₙ is an approximation for the minimum expected Rₐ, and Rₘₐₓ is an approximation for the maximum expected Rₐ.
6. A set of voltages V(t) is calculated for each entry in Rest as:
   a.

$$V ( t ) = \{ \vert {V}_{we - ie} ( t ) - I ( t ) * r \text{\textbar :} r ∈ {R}_{est} , {T}_{1} \leq t \leq {T}_{3} \}$$

7. Find the local maximum for each set of voltages for each entry in Rₑₛₜ
8. Set Ra equal to the element of Rₑₛₜ that generates the minimum local maximum
9. Vpp can be calculated by the following equation, where T₄ is the preceding zero crossing point: a.

$$V pp == \vert {V}_{we - ie} ( {T}_{2} ) - I ( {T}_{2} ) * Ra \vert - \vert {V}_{we - ie} ( {T}_{4} ) - I ( {T}_{4} ) * Ra \vert$$

In one configuration, the algorithm includes the following steps:
1. Two time points, T₅ and T₂, are set at the most previous current zero-crossing and immediately preceding zero-crossing, respectively. Additional time points are set prior to and after each of these initial time points as a function of a sampling period Tₛ. In total, there will be 6 time points in the set: T={T₁, T₂, T₃, T₄, T₅, T₆}, where T₁=T₂−Tₛ, T₃=T₂+Tₛ, T4=T5−Tₛ, and T₆=T₅+Tₛ.
2. V_{we-ie}(t) is sampled at each time point in the set, producing a set of voltages:

$$V = \{ {V}_{1} , {V}_{2} , {V}_{3} , {V}_{4} , {V}_{5} , {V}_{6} \} ,$$

$$where$$

$${V}_{n} = {V}_{we - ie} ( {T}_{n} ) .$$

3. I(t) is sampled at each time point in the set from the device commands to output, or sampled from an output current monitor, producing a set of currents:

$$I = \{ {I}_{1} , {I}_{2} , {I}_{3} , {I}_{4} , {I}_{5} , {I}_{6} \} ,$$

$$where$$

$${I}_{n} = I ( {T}_{n} ) .$$

4. A set of values for access resistance, Rₑₛₜ, is calculated using the last known value for Ra, Ra₀, and a delta value, δ_{Ra}. The value Ra₀ may come from an estimate, prestimulation measurement, or the last dynamic measurement, i.e. the previous result of this algorithm. In total, there will be 3 resistance values in the set: Rₑₛₜ={Ra₁, Ra₂, Ra₃}, where Ra₁=Ra₀−δ_{Ra}, and Ra₃=Ra₀+δ_{Ra}.
5. Calculate a set of relative slopes in the sampled voltages bounded by T₃ and T₁ (or T₆ and T₄) for each resistor value as follows:
   a.

$$S = \{ \vert ( {V}_{3} - {I}_{3} * r ) - ( {V}_{1} - {I}_{1} * r ) \text{\textbar :} r ∈ {R}_{est} \}$$

6. Set Ra equal to the element of r that generates min{S}. This access resistance will then be used in subsequent calculations and future determinations for Ra (setting Ra₀=Ra for the preceding algorithm)
7. Calculate the peak to peak voltage at the current zero-crossing points as follows:
   a.

$$V pp = \vert {V}_{2} - {I}_{2} * Ra \vert + \vert {V}_{5} - {I}_{5} * Ra \vert$$

In some configurations, the algorithm for a low frequency, biphasic waveform includes the following steps:
1. A set of 8 time points is defined as follows across the biphasic waveform:
   a. T_{0,6}=current zero-crossing time, where T₀ is from a previous cycle and T₆ is from the present cycle
   b. T_{1,7}=beginning of first phase current plateau, where T₁ is from a previous cycle and T₇ is from the present cycle
   c. T₂=end of first phase current plateau
   d. T₃=current zero-crossing time
   e. T₄=beginning of second phase current plateau
   f. T₅=end of second phase current plateau
2. V_{we-ie}(t) is sampled at each time point in the set, producing a set of voltages:

$$I = \{ {I}_{0} , {I}_{1} , {I}_{2} , {I}_{3} , {I}_{4} , {I}_{5} , {I}_{6} , {I}_{7} \} ,$$

$$where$$

$${I}_{n} = I ( {T}_{n} ) .$$

3. I(t) is sampled at each time point in the set from the device commands to output, or sampled from an output current monitor, producing a set of currents:

$$V = \{ {V}_{0} , {V}_{1} , {V}_{2} , {V}_{3} , {V}_{4} , {V}_{5} , {V}_{6} , {V}_{7} \} ,$$

$$where$$

$${V}_{n} = {V}_{we - ie} ( {T}_{n} ) .$$

4. Cdl for the the first and second phase is determined from Cdl=Q/(V3−V0) and Q/(V6−V3), respectively, where Q is known from the device commands to output or from an output current monitor and the phase time.
5. Ra for the first and second phase is determined from Ra=(V2−V4)/(I2−I4) and (V7−V5)/(I7−I5)
6. Vcdl (Vpp elsewhere) for the first and second phase is determined from Vcdl=V3−V0 and V6−V3, respectively

The previously described algorithms are capable of assessing Ra and Vpp, and thereby indirectly the capacitance of the working electrode, once every half period. In some configurations, it would be desirable to assess these parameters every half period. In some configurations, it may be desirable to assess these parameters less frequently, such as every 1, 1.5, 2, 2.5, 3, 3.5, 4, 4.5, 5, 5.5, 6, 6.5, 7, 7.5, 8, 8.5, 9, 9.5, 10, or greater periods. In some configurations, it may be desirable to assess these parameters on demand for evaluation by an interested party, such as a patient, physician, device representative, or other parties, or at specific times, such as before device powers off, after device powers on, and for some set period. In some configurations, it may be desirable to observe how these parameters vary over time, including over the total runtime, total implantation time, total number of periods, or other time windows of interest. In some configurations, this algorithm could be applied in post-processing, e.g., not in real-time, such as upon inspection by a device programmer or physician.

In some configurations, it would be desirable to use these measured parameters to modify the delivered waveform. The following example illustrate using such parameters to modify the delivered waveform by the simulation engine or as a safety mechanism or mitigation mechanism to assure proper operation of the stimulation engine.

Example 1a: To assure that electrode is not used over capacity control current based upon real-time voltage across capacitor, that is limit current so that Vcdl does not increase beyond limit.

Example 1b: To assure that electrode is used at maximum capacity vary bias to maximize Cdl.

Example 1c: To assure maximum delivery of bipolar currents use CDL of each WE in pair and split bias between the two contacts to equalize the contact capacity and at a higher level sevro the total bias to achieve maximum combined CDL and capacity.

Example 2a: Use real-time RA measure to get an indication of surrounding tissue health and limit current based upon this parameter.

Example 2b: Another option is to duty cycle current over hours or days assuming the tissue will recover. That rate may be due to profusion rate of surrounding tissues.

Example 3: Modify waveform morphology (current profile & duration) to optimize charge and amplitude and bias to deliver efficacious stimulation with the minimal impact to the electrode and surround tissues by maximizing or minimizing CDL and then using RA as a tissue health indicator.

Each multiplexer (e.g., VRE, SRCA MUX, SRCB MUX, etc.) of the stimulation engine may be monitored, as well, to assure proper operation. For example, VOS (FIG. 12) may be monitored to check the current to and from each multiplexer. Out of range values (e.g., excessive current flowing to the power supplies of the multiplexers) can indicate a failure of one or more channels of (or the entire) multiplexer.

The WEMX mechanism refers to two multiplexers (e.g., SRCA MUX, SRCB MUX of FIG. 12) are used to mitigate the risk of multiplexer failure, and low level ground leakages. By routing therapeutic current waveforms through two multiplexers before reaching a selected electrode (and patient), both multiplexers would need to fail to cause low level ground leakages.

The CBSW mechanism refers to utilizing capacitor bypass switches ULFSWA and ULFSWB to perform a self-test on the stimulation engine circuitry. For example, in one embodiment, the capacitor bypass switches ULFSWA or ULFSWB are activated or deactivated while the multiplexers SRCA MUX, SRCB MUX short the selected working electrodes together to create a test loop. The stimulation engine then tries to send a DC signal through the test loop to determine if the capacitor bypass switches and bypass capacitors, and other circuits of the loop, are operating properly.

The MCU mechanism refers to providing redundant MCUs (e.g., Main MCU and Safety MCU), as discussed above. In some instances each MCU may maintains separate peripherals to improve reliability, or in some cases MCUs may share peripherals or share data from those peripherals.

The IESW mechanism refers to providing multiple series switches to the indifferent electrode. Such fault tolerance assures that the patient is not exposed to the Vic signal on the can during AC mode operation.

The VSR mechanism refers to providing multiple series resistors to the Vref signal. Such fault tolerance assures that in the extremely rare event of a Vref failure resulting in current flowing out of an input, the series resistors limit such current.

The VRMX mechanism refers to providing a separate multiplexer VRE to access and sense a reference voltage Vre on a selected electrode E01-E16. The multiplexer is configured to disconnect a Vref amplifier from the working electrodes (e.g., selected electrode E01-E16 ) when it is not in use.

The BCAP mechanism, as described in additional detail herein, refers to dual blocking capacitors CBA, CBB (one for each amplifier SRCA, SRCB) that guarantee that the can may be switched into the stimulation path during AC stimulation mode to assure that no DC current is provided during the AC stimulation mode.

The ESD mechanism refer to the stimulation engine including one or more electrostatic discharge (ESD) pathways to sink unwanted electrostatic energy. Each ESD pathway may include a diode to the internal voltage rail and another diode to the internal ground rail. Where the voltage between the rails is limited by a clamping diode of a selected voltage. This configuration of diodes is referred to as steering diode configuration and is required to accommodate both bipolar and unipolar operation within a variable voltage range such that the parasitic diodes on the output circuits are protected where it is assumed that the operation voltages of those circuits is tied to the variable voltage range. Each steering diode can be converted to a series pair so if one diode fails the other will still be operational. As a further circuit optimization if multiple channels exist the 2^{nd} series does may be grouped together into one diode device to reduce the component count.

The CALR mechanism refers to providing an calibration load (e.g., CAL of FIG. 12). The calibration load may be used in conjunction with the current generators SRCA, SRCB, TRIM-A, TRIM-B and the VIE current sense monitor to calibrate the current generators.

The WEAM mechanism refers to a WEAM monitor that compares the output voltages of the stimulation engines two amplifiers CSA, CSB. Because the stimulation engine operates in a bipolar state, a controller expects to see symmetrical values (e.g., the voltage of one amplifier should equal the same or opposite voltage of the other amplifier), or value that differ by only a small, known offset.

Furthermore, the ACALₓ mechanism refers to the current outputs being calibrated by switching in a calibration load CAL into the stimulation engine's output path and monitoring the current flowing through the calibration load (e.g., with a current sensor Iie). By determining a difference between the measured current and the programmed or desired current, an offset or calibration adjustment may be made to the stimulation engine. For example, the input to a DAC may be increased or decreased by an offset value to compensate for such differences.

Double switches for redundancy on the capacitor CBA, CBB bypass switches ULFSW can provide an additional layer of security. Series switches IESW1, IESW2 to the IE can also provide an additional layer of safety to protect against AC transmission to the stimulation engine's indifference electrode (e.g., metallic housing, or can). Two high impedance resistance resistors VR1, VR2 may be provided to protect against unlikely amplifier failure. A multiplexer VRE can be utilized to disable the electrode multiplexers SRCA MUX, SRCB MUX if a failure in the Vref system is detected. An amplifier calibration load can be used to calibrate voltage amplifiers in conjunction with current generators. For example, amplifiers directing current to the IE should be accurately outputting the desired currents. When the stimulation engine is powered up, it can switch in a calibration resistor to route current into the calibration resistor, to the IE, and to the current sensor (Iie) to make sure that the amplifier path is working properly. Such circuitry can also function as a discharge path for charge recovery. When in AC mode, two forms of charge recovery are possible. During active recovery, as discussed above, a pulse of a desired amplitude is output and then a fractional pulse is sent for a longer duration. For example, a 1 μs stimulation pulse may be delivered and then an 8 μs recovery pulse may be applied. The same amount of current is sent in opposite directions. During passive recovery, a stimulation pulse is sent to the electrode, and then a capacitor or resistor is switched in to the circuit to recover.

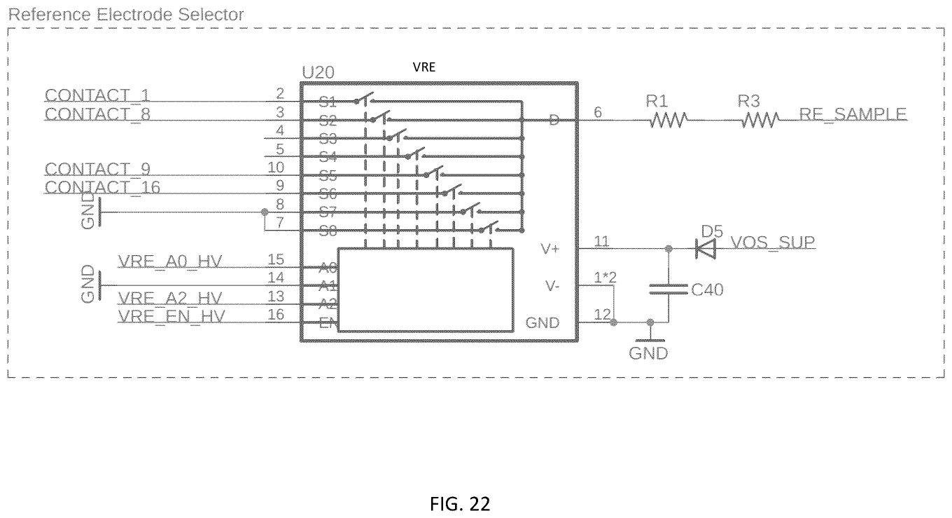

FIG. 22 illustrates one embodiment of an output multiplexer U20. The multiplexer U20 may be used by the stimulation engine to select an electrode to receive a stimulation pulse, an electrode to form a return path for the stimulation pulse, and/or an electrode to serve as a reference electrode. The multiplexer U20 may be employed as any one or more of VRE, SRCA MUX. SRCB MUX, IESW1 and/or IESW2 of FIG. 12. In addition, the multiplexer U20 includes a back biasing diode D5 the prevents back biasing to the multiplexer. Back biasing could result in undesirable DC current flowing to the multiplexer; therefore, the back biasing diode D5 assures that the multiplexer U20 may not enter into a state in which it could possibly become back biased. The back biasing diode D5 may be provided with any one or more of the components VRE, SRCA MUX, SRCB MUX, IESW1 and/or IESW2 of FIG. 12.

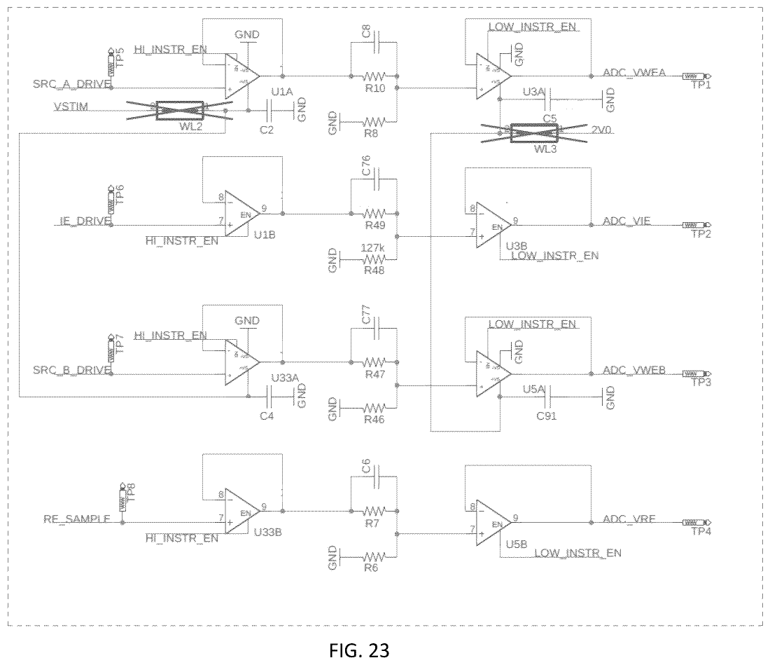

FIG. 23 illustrates various instrumentation amplifiers U1A, U1B, U33A, U33B, U3A, U3B, U5A, U5B, that may be used to measure the Vref, Vwe1, Vwe2 and IE voltages of the stimulation engine. The voltages are read by one or more of the Main MCU and the Safety MCU of FIG. 12. Test points TP1-TP4 correspond to buffered Vre, VweA, VweB and Vie voltages.

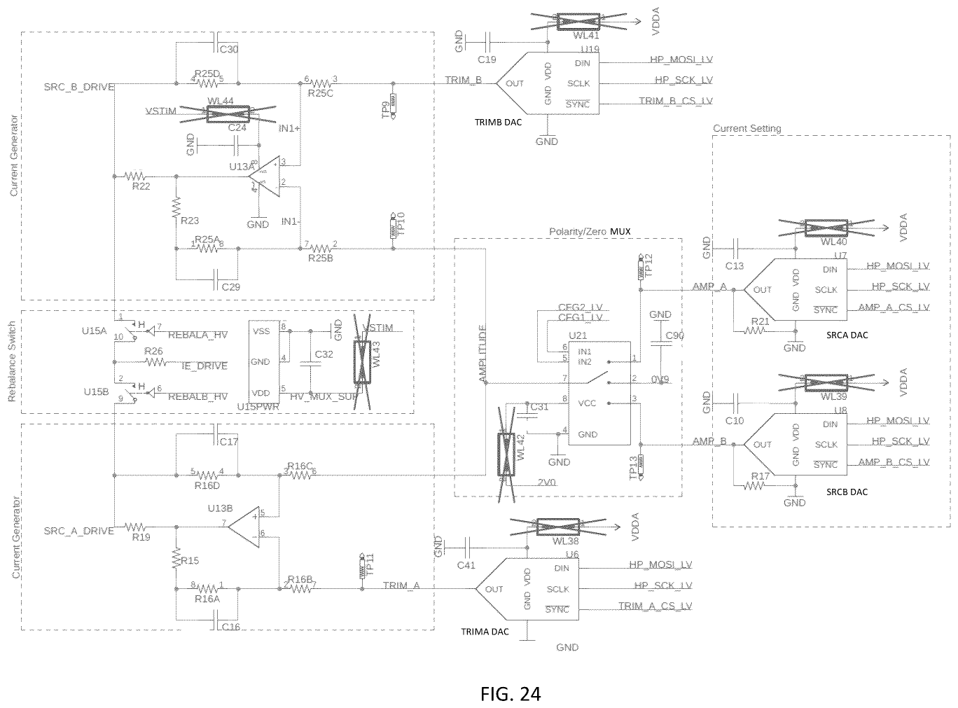

FIG. 24 illustrates various current generators, rebalance switches, trim DACs, a polarity/zero circuit, and a current setting circuit. The current setting circuit has two DACs U7 and U8 (corresponding to SRCA DAC and SRCB DAC of FIG. 12). Each DAC is set to a different fixed output voltage level. The switch U21 (corresponding to CFG of FIG. 12) of the polarity/zero circuit is able to quickly switch between the two DAC outputs, and sends the selected DAC output to the amplifiers U13A, U13B (corresponding to SRCA and SRCB of FIG. 12). The switch U21 is able to switch between the DAC outputs much faster than the DAC would be able to change its output in response to a changing input. Therefore, the switch U21 allows the stimulation engine to provide a very quick AC pulse (e.g., 100 Hz, 250 μs pulse width, etc.). Two trim DACs U19, U6 (corresponding to TRIMB DAC and TRIMA DAC of FIG. 12) are able to trim the amplifiers in a variety of ways. For example, each trim DAC U19, U6 can be used to set a desired bias current to each current source when in ULF/DC mode. In addition, each trim DAC U19, U6 can also compensate for any offset voltages present in the op amps that are used to generate the currents (e.g., U13A, U13B). Furthermore, rebalance switches U15A, U15B (corresponding to RBSWA and RBSWB of FIG. 12) can be used to disconnect the IE from the can and use it as a virtual ground to recover charge off of the capacitors when in AC mode. In ULF/DC mode, the IE may be used for the bias current, which also serves as a virtual ground.

The stimulation engine of FIG. 12 includes an indifferent electrode and instrumentation to apply and control an active voltage on the stimulation engine's indifferent electrode. A digital-to-analog converter VIE DAC is controlled to generate an indifferent electrode voltage (or IE drive or virtual ground). The VIE DAC output passes through a buffer or voltage follower DRV. The stimulation engine can sense the current to the indifferent electrode with a current monitor Iie. An amplifier may boost the voltage, which is then analyzed by one or both processors Main MCU, Safety MCU, to make sure that it is in range.

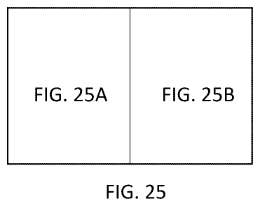

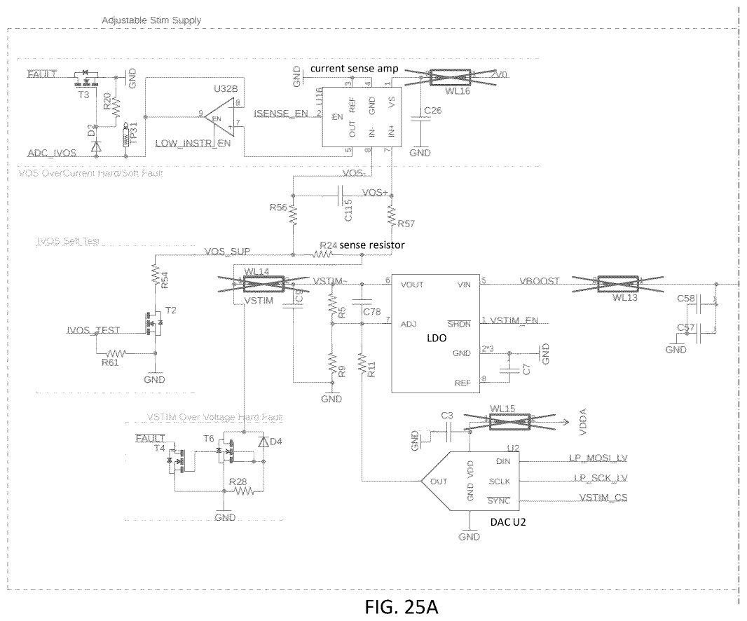

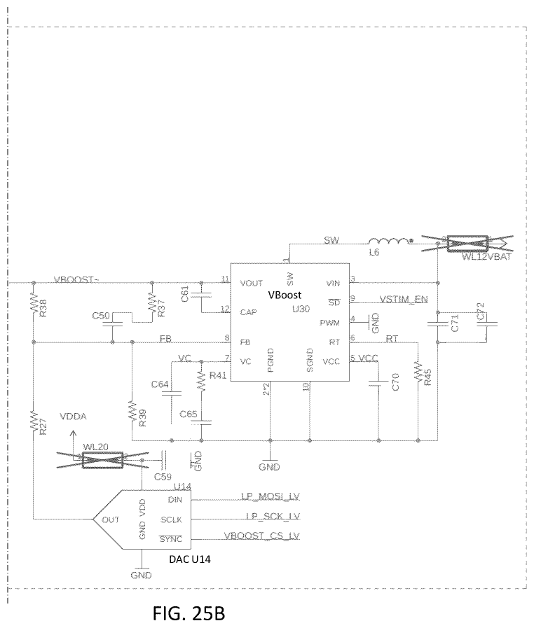

FIG. 25 illustrates one embodiment of an adjustable stimulation power supply with fault detection employed by the stimulation engine of FIG. 12. The illustrated circuit may be used to sense the current used to drive the stimulation engine's multiplexers. A boost supply generates a high voltage signal Vboost for tissue stimulation. The Vboost signal is down regulated to generate a Vstim signal. Vboost is generated at a switching regulator, which can be noisy. Noise is lowered by adding the secondary regulator to generate Vstim. A DAC U2 is used to control the Vstim signal in order to optimize power. A fault detector (VSTIM Over Voltage Hard Fault) can determine if the Vstim signal has exceeded the desired level. If so, the fault detector turns off the Vstim signal. The mulitplexers are supplied with VOS+ and VOS− signals, and current is sensed at resistors R56, R57 and amplified at current sense amplifier U16. A comparator U32 can determine if the Vstim signal is too high, or not at the desired level. If so, a fault is sent to the microprocessor Main MCU, Safety MCU. The microprocessor can also be used to create a fault to test the fault detection circuity. For example, the microprocessor can set the regulator output very high to confirm that fault detection circuitry is operating correctly.

The foregoing description and examples has been set forth to illustrate the disclosure according to various embodiments and are not intended as being unduly limiting. The headings provided herein are for organizational purposes only and should not be used to limit embodiments. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. References cited herein are incorporated by reference in their entirety.

While the methods and devices described herein may be susceptible to various modifications and alternative forms, specific examples thereof have been shown in the drawings and are herein described in detail. It should be understood, however, that the embodiments disclosed should cover modifications, equivalents, and alternatives falling within the spirit and scope of the various embodiments described herein and the appended claims.

Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially.

The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed.

The various illustrative logical blocks, modules, processes, methods, and algorithms described in connection with the embodiments disclosed herein can be implemented as electronic hardware, computer software, or combinations of both. To clearly illustrate this interchangeability of hardware and software, various illustrative components, blocks, modules, operations, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The blocks, operations, or steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, an optical disc (e.g., CD-ROM or DVD), or any other form of volatile or non-volatile computer-readable storage medium known in the art. A storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular embodiment.

The methods disclosed herein may include certain actions taken by a practitioner; however, the methods can also include any third-party instruction of those actions, either expressly or by implication. For example, actions such as “positioning an electrode” include “instructing positioning of an electrode.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example +5%, +10%, +15%, etc.). For example, “about 1 hour” includes “1 hour.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure. The phrase “at least one of” is intended to require at least one item from the subsequent listing, not one type of each item from each item in the subsequent listing. For example, “at least one of A, B, and C” can include A, B, C, A and B, A and C, B and C, or A, B, and C.

What is claimed is:

1. A neuromodulation device configured to operate in a plurality of waveform generation modes, comprising:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode; and
an indifferent electrode configured to provide a return path for the alternating current, the direct current, or both;
wherein the control unit is configured to detect at least one failure event and prevent, alter, or stop operation of the bipolar current generator in response to the detected at least one failure event.

2. The neuromodulation device of claim 1, wherein the failure event comprises an actual stimulating current being unequal to a desired stimulating current, wherein the actual stimulating current is the alternating current or the direct current.

3. The neuromodulation device of claim 1, wherein the failure event comprises a monitored current signal from a power supply to the switching unit exceeding an expected current amount.

4. The neuromodulation device of claim 1, wherein the failure event comprises a real time determination that at least one of a resistance or capacitance of the at least one working electrode is not equal to an expected resistance or expected capacitance of the at least one working electrode.

5. The neuromodulation device of claim 1, wherein the failure event comprises a real time determination that a peak-to-peak voltage of the at least one working electrode is above an expected voltage of the at least one working electrode.

6. The neuromodulation device of claim 1, wherein the failure event comprises a blocking capacitor is not properly functioning.

7. The neuromodulation device of claim 1, wherein the direct current comprises an anodic current and a cathodic current, and wherein the failure event comprises the anodic current not being (1) equal to and (2) opposite in sign to the cathodic current.

8. The neuromodulation device of claim 1, wherein the direct current comprises an anodic current and a cathodic current, and wherein the failure event comprises the anodic current not being (1) less than a threshold amount different from and (2) opposite in sign to the cathodic current.

9. The neuromodulation device of claim 1, wherein the failure event is any of the failure events described herein.

10. A neuromodulation method, comprising:
providing:
a power source;
a control unit in communication with the power source;
a bipolar current generator in communication with the control unit and an input of a switching unit; and
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode; and
delivering a direct current to the working electrode during a second waveform generation mode;
detecting at least one failure event; and
preventing, altering, or stopping operation of the bipolar current generator in response to detecting at least one failure event.

11. A neuromodulation device configured to operate in a plurality of waveform generation modes, comprising:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode; and
an indifferent electrode configured to provide a return path for the alternating current, the direct current, or both;
wherein the control unit is configured to monitor a current flowing to a power supply of the switching unit and to deactivate the bipolar current generator when the monitored current violates a threshold condition.

12. The neuromodulation device of claim 11, wherein violating a threshold condition corresponds to the monitored current exceeding a threshold level.

13. The neuromodulation device of claim 11, wherein violating a threshold condition corresponds to the monitored current falling below a threshold level.

14. The neuromodulation device of claim 11, wherein the switching unit comprises a multiplexer.

15. The neuromodulation device of claim 11, further comprising a back-biasing diode in communication with at least one output of the switching unit, wherein the back-biasing diode is configured to prevent back-biasing of the switching unit at least one output.

16. A neuromodulation method, comprising:
providing:
a power source;
a control unit in communication with the power source;
a bipolar current generator in communication with the control unit and an input of a switching unit; and
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode;
delivering a direct current to the working electrode during a second waveform generation mode;
monitoring a current flowing to a power supply of the switching unit; and
deactivating the bipolar current generator when the monitored current violates a threshold condition.

17. A neuromodulation device configured to operate in a plurality of waveform generation modes, comprising:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode;
an indifferent electrode configured to provide a return path for the alternating current, the direct current, or both; and
a bipolar current generator calibration unit comprising a calibration load and a calibration load switch, wherein the control unit is configured to activate the calibration load switch to direct current from the bipolar current generator to the calibration load, measure the current directed to the calibration load, and calibrate the bipolar current generator in response to the measured current.

18. The neuromodulation device of claim 17, wherein the calibration load comprises a resistor.

19. The neuromodulation device of claim 17, further comprising a current sensor configured to measure the current directed to the calibration load.

20. The neuromodulation device of claim 17, wherein calibrating the bipolar current generator comprises adjusting the value of a control signal communicated to bipolar current generator.

21. A neuromodulation method, comprising:
providing:
a power source;
a control unit in communication with the power source;
a bipolar current generator in communication with the control unit and an input of a switching unit; and
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode;
delivering a direct current to the working electrode during a second waveform generation mode;
providing a bipolar current generator calibration unit comprising a calibration load and a calibration load switch;
activating the calibration load switch to direct current from the bipolar current generator to the calibration load;
measuring the current directed to the calibration load; and
calibrating the bipolar current generator in response to the measured current.

22. A neuromodulation device configured to operate in a plurality of waveform generation modes, comprising:
a power source;
a control unit in communication with the power source, wherein the control unit comprises a power controller and a trimming controller;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode; and
an indifferent electrode configured to provide a return path for the alternating current, the direct current, or both;
wherein the power controller is configured to provide a power control signal corresponding to a desired current output level to the bipolar current generator, wherein the trimming controller is configured to provide an adjustment signal to the bipolar current generator, and wherein the current generator is further configured to deliver the alternating current in the first waveform generation mode or the direct current in the second waveform generation mode in response to the power control signal and the adjustment signal.

23. The neuromodulation device of claim 22, wherein the bipolar current generator comprises at least one amplifier comprising first and second terminals, and wherein the first terminal is in electrical communication with the power controller and wherein the second terminal is in electrical communication with the trimming controller.

24. A neuromodulation method, comprising:
providing:
a power source;
a control unit in communication with the power source, wherein the control unit comprises a power controller and a trimming controller;
a bipolar current generator in communication with the control unit and an input of a switching unit; and
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode;
delivering a direct current to the working electrode during a second waveform generation mode; and
determining a power control signal corresponding to a desired current output level to the bipolar current generator;
determining an adjustment signal to the bipolar current generator;
delivering the alternating current in the first waveform generation mode or the direct current in the second waveform generation mode in response to the power control signal and the adjustment signal.

25. A neuromodulation device configured to operate in a plurality of waveform generation modes, comprising:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode;
an indifferent electrode configured to provide a return path for the alternating current, the direct current, or both; and
a protection mechanism configured to prevent electrical damage to the neuromodulation device from energy from an external source.

26. The neuromodulation device of claim 25, wherein the protection mechanism does not include capacitors

27. The neuromodulation device of claim 25, wherein the external source is an electrocautery device or a defibrillator.

28. The neuromodulation device of claim 25, wherein the protection mechanism comprises at least one positive temperature coefficient (PTC) device and a Zener diode.

29. The neuromodulation device of claim 28, wherein the Zener diode is electrically in series with at least one PTC device.

30. A neuromodulation method, comprising:
providing:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode;
delivering a direct current to the working electrode during a second waveform generation mode; and
preventing electrical damage to one or more of the power source, the control unit, the bipolar current generator, or one or more of the plurality of electrodes from energy from an external source.

31. A neuromodulation device configured to operate in a plurality of waveform generation modes, comprising:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
wherein the current generator is configured to deliver alternating current to the at least one working electrode during a first waveform generation mode, and wherein the current generator is further configured to deliver a direct current to the working electrode during a second waveform generation mode; and
an indifferent electrode configured to provide a return path for the alternating current, the direct current, or both;
wherein the control unit is configured to determine at least one parameter of an electrode in real time and modify operation of the current generator in response to detecting a change in the at least one parameter of the electrode that is greater than a threshold amount.

32. The neuromodulation device of claim 31, wherein parameter of the electrode is a series access resistance (Ra) or a double layer capacitance (Cdl).

33. The neuromodulation device of claim 32, wherein the control unit is configured to determine the at least one parameter of the electrode in real time by using a test rectangular biphasic current pulse and a potential measured between a working and indifferent electrode.

34. The neuromodulation device of claim 31, wherein the parameter of the electrode is a series access resistance (Ra) or a cyclic peak-to-peak voltage (Vpp).

35. The neuromodulation device of claim 34, wherein the control unit is configured to determine the at least one parameter of the electrode in real time by using a set of points sampled from a potential measured between a working and indifferent electrode and an instantaneous stimulus current.

36. The neuromodulation device of claim 31, wherein the control unit is configured to use the at least one parameter of the electrode to control the device according to any of the methods or examples provided herein.

37. A neuromodulation method, comprising:
providing:
a power source;
a control unit in communication with the power source;
a bipolar current generator in communication with the control unit and an input of a switching unit;
a plurality of electrodes, each in communication with a unique output of the switching unit, wherein the switching unit is configured to provide electrical communication between the bipolar current generator and a selected one of the plurality of electrodes in response to a control signal from the control unit;
delivering alternating current to the at least one working electrode during a first waveform generation mode;
delivering a direct current to the working electrode during a second waveform generation mode;
determining at least one parameter of an electrode in real time; and
modifying operation of the current generator in response to detecting a change in the at least one parameter of the electrode that is greater than a threshold amount.

38. The neuromodulation device of any of the preceding claims, wherein the direct current comprises ultra low frequency current.

39. The neuromodulation device or method of any of the preceding claims, wherein the ultra low frequency currents are less than about 5 Hz.

40. The neuromodulation device or method of any of the preceding claims, wherein the ultra low frequency currents are less than about 2 Hz.

41. The neuromodulation device or method of any of the preceding claims, wherein the ultra low frequency currents are less than about 1 Hz.

42. The neuromodulation device or method of any of the preceding claims, wherein the alternating current is high frequency alternating current.

43. The neuromodulation device or method of any of the preceding claims, wherein the high frequency alternating current is at least about 1 kHz.

44. The neuromodulation device or method of any of the preceding claims, wherein the alternating current is between about 5 Hz and about 1 kHz.

45. The neuromodulation device or method of any of the preceding claims, wherein the power source comprises a battery.

46. The neuromodulation device or method of any of the preceding claims, wherein the control unit comprises a first control unit and a second control unit configured to run independent algorithms.

47. The neuromodulation device or method of any of the preceding claims, wherein the device is configured to measure the offset current when the device is in the second waveform generation mode.

48. The neuromodulation device or method of any of the preceding claims, wherein the device is configured to measure cyclic Vpp of the at least one working electrode.

49. The neuromodulation device or method of any of the preceding claims, further comprising a virtual ground configured to be operably connected to the indifferent electrode where the virtual ground can be set to any level to minimize power dissipation.
